(19) United States
(12) Patent Application Publication
(10) Pub. No.: US 2011/0129488 A1
(43) Pub. Date: Jun. 2, 2011

(54) IMMUNOTHERAPY COMPOSITIONS FOR THE TREATMENT AND PREVENTION OF AMYLOIDOSIS

(75) Inventors: Josette-Nicole Mahy Gehenne, Barcelona (ES); Manuel Rodriguez Allue, Barcelona (ES); Jordi Riba Casellas, Barcelona (ES); Josep Puigdollers Masallera, Barcelona (ES); Pau Llobera Mahy, Baleares (ES); Josep Maria Closa Boixeda, Barcelona (ES); Artur Font Utset, Barcelona (ES); Juan Llobera Mahy, Barcelona (ES); Gemma Vivet Dordal, Barcelona (ES); Juan Maria Mascort Boixeda, Barcelona (ES); Marco Pugliese, Barcelona (ES)

(73) Assignee: MEDIVET PHARMA, S.L., Barcelona (ES)

(21) Appl. No.: 13/056,459

(22) PCT Filed: Jul. 28, 2009
(86) PCT No.: PCT/EP09/59772

§ 371 Date: Feb. 16, 2011

(30) Foreign Application Priority Data
Jul. 29, 2008 (EP) 08161384.6

(51) Int. Cl. A61K 39/00; A61P 25/24; A61P 25/28; C07K 16/18; G01N 33/53

(52) U.S. Cl. 424/185.1; 530/387.9; 436/501

(57) ABSTRACT

The invention relates to immunogenic peptide sequences and antibodies against said immunogenic agents for treating, preventing and/or diagnosing in human and non-human mammals a disease which is characterized by amyloid deposition, including involutive depression, confusional syndrome, dysthymia and cognitive Dysfunction Syndrome (CDS) in non-human mammals and different amyloidosis, including Alzheimer's disease in humans. Moreover, the invention provides diagnostic methods for the above diseases using the antibodies of the invention.

The present invention relates to peptide sequences and antibodies raised against these peptide sequences as agents for curing amyloid-related diseases in patients and, in particular, cognitive dysfunction-related diseases in non-human mammals. This invention regards to the field of human and veterinary diseases, especially for attending companion, domestic and livestock animals.

### BACKGROUND ART

With the presence of improved standard of veterinary care, veterinarians are seeing and treating more geriatric animals in their clinical practice. In most countries, the number of companion animals is rapidly increasing. For example, data from 1997 calculate in approximately 52.5 million the number of pet dogs and 57 millions of cats in the United States; and in 7.3 million (14%) of dogs and 6.27 million (11%) of cats the 11 year-old or older. Presently, these numbers are some 20% higher. The same occurs in Europe, where live some 38 million of dogs and 36.6 million of cats and a 25-30% of these animals are considered old.

Companion, domestic and livestock animal ageing represent a complex biological process, characterized by a progressive modification of tissues and cells with a gradual loss of adaptive capacity, and it is associated with progressive and irreversible changes in the body systems. Presently, the pathogenesis of normal and altered ageing is viewed as a multi-factored event in which oxidative stress plays a pivotal role. Although these changes are usually considered individually, the elderly animal is seldom afflicted with a single disease but rather with varying degrees of organ dysfunction. Pet owner complaints include cognitive changes that describe geriatric behavioral changes not solely due to a general medical condition such as infection, organ failure or neoplasm. These behavioral changes fall mostly into four main categories: a) loss of cognition and recognition, b) loss of housetraining, c) disorientation and d) changes in the sleep-wake cycle.

A large number of studies demonstrated that cognitive ability in non-human mammals decreases with age following specie-specific patterns, especially in dogs. Based on the predominance of affective or cognitive dysfunctions, a diagnosis of Dysthimia, Cognitive Dysfunction Syndrome (CDS), Involutive depression or Confusional Syndrome is done.

The typical clinical sign of dysthymia in animals is the loss of the ability to evaluate the relationship between a length of a passage and your own body. The dysthymic animal tends to force the way and may well remain trapped during hours moaning. Any attempt to foreign aid may trigger aggressive responses. Other clinical signs of dysthymia are going to wrong side of doors; appearing to forget previously learned tasks getting stuck in corners and behind furniture.

The term cognitive dysfunction syndrome (CDS) means the age-associated decline in the cognitive abilities of an animal that cannot be attributed to an unrelated general medical condition such as neoplasia, infection, or organ failure. Symptoms of cognitive dysfunction syndrome include, but are not limited to, altered interaction with family members, changes in sleep-wake cycle, decreased activity level, memory loss and inappropriate elimination. The Involutive depression includes depression, lethargy, and symptoms of cognitive dysfunction syndrome. The Confusional syndrome includes daze, hesitation, wandering aimlessly and symptoms of cognitive dysfunction syndrome.

In all these clinical presentations, reports of retraction of cerebral gyri and widening of sulci together with an increase in ventricular volume are described. Microscopically, lipofuscin storage, polyglucosan body, specific neuronal loss and β-amyloid (Abeta) plaque deposition are also common. In addition, these changes frequently occur in aging animals.

The cause of CDS is unknown. Studies have shown that its symptoms increase with age, and many pathological changes occur in aging dogs and cats that can theoretically lead to CDS. One such change, which has been correlated with CDS in dogs, is the formation of β-amyloid plaques in the Central Nervous System (CNS). Another change is the decline in activity of several neurotransmitters, including serotonin, acetylcholine, norepinephrine, and dopamine. Still other potential causes of CDS include, but are not limited to, elevated monoamine oxidase B (MAO-B) activity located in astroglia, and oxidation of central nervous system lipid membrane.

Nowadays the diagnosis of CDS is performed based on an indirect evaluation of the dog behavior through a formal questionnaire to the owner (Pugliese et al., “Severe cognitive impairment correlates with higher cerebrospinal fluid levels of lactate and pyruvate in a canine model of senile dementia” (2005), Prog. Neuropsychopharmacol. Biol. Psychiatr—2005, 29:603-610). This new cognitive test allows, in a reduced time, the discrimination between light and severe cognitive impaired behavior in companion dogs, ruling out the need of a specific strain to be housed during years and trained in controlled conditions. Our test, filled out by a veterinary neurologist, consisted of 16 items. For each item, score 1 indicates the normality of the specific behavior, and score 2, 3, 4 and 5 the degree of abnormal behavior. The final total score reflects the cognitive status of the animal: a 16-79 score range was allows the classification of the animals into three groups: 1) young control animals (YC); 2) dogs with light cognitive deficits (LCD); 3) dogs with severe cognitive deficits (SCD). In addition, cerebrospinal fluid (CSF) lactate, pyruvate and potassium concentrations relate with the degree of cognitive deficit. This reflects a specific relationship between availability of energy substrates and canine age-related cognitive impairment in which astroglia plays a key role and relates with MAO-B altered activity.

As in dogs, felines present a variety of behavioral changes with aging that are consistent with specific cognitive dysfunction still to be fully characterized. These signs include: inappropriate urination, including spraying and house soiling, aggression towards people and intraspecies aggression, overactivity, excessive vocalization mainly during the night, altered-sleep wake cycle, fear or anxiety, ingestive behavior, scratching furniture. In elder cats two cognitive diseases related to aging may be diagnosed, an Involutive Depression and a Dysthymia of the aging cat.

A major part of research in animal cognitive function and neuropathology is largely dedicated to elucidation of the molecular events responsible of their cognitive impairment and neuronal loss. A wide variety of age-related changes have been described in the nervous system of many non-human species. The most consistently observed brain lesions in canine, feline and polar bear mostly reflects diffuse deposits of Abeta (β-amyloid) peptide and their inability to form paired helical filaments (PHF), develop neurofibrillary tangles and mature neuritic plaques and to activate microglia. Together with the age-related cognitive decline, these brain lesions represent the major factors implicated in neurodegeneration. Together with canine aging, Abeta deposition in its early phase participates of the specific neuronal loss and then matures as a diffuse plaque,—never as a dense-core Abeta plaque—in absence of any infiltrating hyperactive microglia. In these animals, a correlation between the degree of cognitive deficit, four stages (stages I-IV) of the diffuse plaque maturation, and expression of two astroglial markers, S100beta and GFAP has been established.

The Abeta protein, a 4 kDa peptide, 42 to 43 amino acids in length, is produced by abnormal cleavage of a transmembrane protein, the amyloid precursor protein (APP) of unknown function, and is highly toxic to neurons. Several studies indicated that APP plays an essential role in the onset of the disease. The presence of β-amyloid (Abeta) plaque caused by anomalous processing of the APP protein constitutes a central event in the pathogenesis of the non-human mammal cognitive deficit, such as dogs and cats. The protein is sequentially cleaved by means of the action of alpha, beta and gamma secretases. In this pathological process, APP is anomalously processed, resulting in insoluble peptides, with 40-42 amino acids (Abeta 1-40 and Abeta 1-42), which tend to aggregate as fibrils and are deposited as diffuse plaques. In these animals, the diffuse plaques present specific characteristics in the way that they are the end product of the pathological process, they do not adopt a beta-pleated-sheet conformation, for what they are Congo red and thioflavine negative, and contain more Abeta1-42 than Abeta1-40. These plaques also contain epitopes within Abeta1-17, Abeta17-24, and Abeta1-28.

Plaque maturation is arrested at the diffuse stage because a specific on-going damaging process develops in these animals without any microglia activation and no extensive astrogliosis. The expression of some neurotrophic factor also presents non-human mammal specificities (Pugliese et al., “Canine cognitive deficit correlates with diffuse plaque maturation and S100beta (−) astrocytosis but not with insulin cerebrospinal fluid level.” Acta Neuropathol—2006, 111:519-528).

Results have shown the formation of diffuse β-amyloid plaques throughout all cortical grey matter layers of canine brain. This process begins when animals are 8 year-old dogs, and increases with age and cognitive deficit severity, following a four-stage maturation process of the diffuse plaques. A positive correlation between plaque density, stage of Abeta deposition and cognitive deficit has also been demonstrated. Plaque formation and maturation were not associated with modifications in synaptic protein expression. Indeed, the active mitogen activated protein kinase (MAPK/ERK-P), p38 kinase (p38-P) expression, and tau hyper-phosphorylation in neighboring cell processes remains unchanged. These results argue for regarding Abeta diffuse plaque formation and tau hyperphosphorylation as independent events, in opposite as what occurs in human aging and Alzheimer's disease (Pugliese et al., Acta Neuropathologica, 2006, 112:175-183).

Unlike in the rodent, the amino acid sequence of Abeta of polar bear, rabbit, cow, cat, sheep, pig, guinea pig, dolphins and Iberian lynx deduced from amplified cDNA is identical to the canine and monkey sequence and these species accumulate β-amyloid plaques with age. In addition to these characteristics these aged non-human mammals exhibit high levels of brain oxidative stress.

Different therapeutic approaches have been selected for cognitive dysfunction research therapeutics in the veterinary field: neuroprotective, restorative or anti-amyloid approaches.

Examples of neuroprotective and restorative approaches are the treatments with selegiline and nicergoline. Selegiline inhibits the monoamine oxidase B (MAO-B), thus reducing oxidative damage and enhancing the activity of dopamine. Besides, nicergoline produces brain vasodilatation, increasing neuronal activity and decreasing neuronal degeneration. All these chronic therapies present severe side effects, such as vomiting, diarrhea or changes in behavior, such as hyperactivity and restlessness.

The amyloid hypothesis (approach) posits that Abeta peptides derived from the proteolytic processing of the transmembrane APP, initiate the process leading to neuronal dysfunction and clinical impairment. Within the anti-amyloid therapeutic approaches, one is based on the identification of small molecules that could inhibit one or another step of amyloid induced cascade in now well under way. Of particular interest are other approaches that attempt to interfere with the aggregation of Abeta 1-42 peptides by decreasing their secretion from neuronal and glial cells or by inhibiting the toxicity that these extracellular aggregates produce on neurons and glial cells and their processes.

The transport of Abeta between CNS and plasma plays a role in the regulation of brain amyloid levels, with Abeta being rapidly transported from cerebrospinal fluid (CSF) to plasma. Therefore of particular interest is the anti-amyloid approach that relies in active vaccination with Abeta peptides that can alter the dynamic equilibrium between the plasma, CSF and ultimately the CNS.

Current evidences suggest that increased plasma Abeta levels following active or passive immunization is due to an active peripheral clearance named as sink effect. The induction or direct plasma addition of anti-Abeta or other antipeptide antibodies serve to quickly and efficiently increase the clearance of CNS Abeta. Generation of this peripheral sink mechanism via induction or administration of antibodies is then useful for treating abnormal Abeta protein accumulation in the CNS, resulting in a net efflux of central Abeta to the periphery for its further rapid elimination.

Head et al. (J. Neuroscience, 2008; 28:3555-66) has described an assay on immunized aged beagles (8.4-12.4 years) with fibrillar Aβ (1-42) formulated with aluminum salt (Alum) for 2.4 years. Although the levels of soluble and insoluble Aβ 1-40 and Aβ 1-42 and the extent of diffuse plaque accumulation was significantly decreased in several cortical regions, the cognitive testing during this time revealed no improvement in measures of learning, spatial attention, or spatial memory.

Thus, it is highly desirable to find successful therapies to improve the quality of life of aging pet and livestock animals suffering from a disease such as in Involutive Depression, Confusional Syndrome, Dysthymia and Cognitive Dysfunction Syndrome (CDS).

### SUMMARY OF THE INVENTION

In a first aspect, the invention relates to a agent for use in the treatment or prevention in a non-human mammal of a disease associated with amyloid deposition wherein said agent comprises a peptide selected from the group of a peptide having a sequence of SEQ ID NO:1 and a functionally equivalent variant thereof.

In a second aspect, the invention relates to a conjugate comprising a peptide having the sequence of SEQ ID NO:3 and an immunologically active carrier wherein the peptide and the carrier are covalently coupled.

In yet another aspect, the invention relates to a composition comprising a first component and a second component selected from the group of
(i) a composition wherein the first component comprises a peptide having a sequence of SEQ ID NO:3 and the second component comprises one or more peptides selected from the group of a peptide having a sequence of SEQ ID NO:10 and a functionally equivalent variant thereof,
(ii) a composition wherein the first component comprises a peptide having a sequence of SEQ ID NO:3 and the second component comprises one or more peptides selected from the group of a peptide having a sequence of SEQ ID NO:10 and a functionally equivalent variant thereof and wherein the peptide forming part of the first component and/or the peptide or peptides forming part of the second component are covalently coupled to an immunologically active carrier and
(iii) a composition comprising a first component comprising a peptide having a sequence of SEQ ID NO:3 and a second component comprising one or more peptides selected from the group of a peptide having the sequence of SEQ ID NO:10 and a functionally equivalent variant thereof wherein both the peptide forming the first component and the peptide or peptides forming the second component are covalently coupled to a single immunologically active carrier.

In further aspects, the invention relates to a conjugate as defined above or to a composition as defined above for use in medicine or for use in the treatment of an amyloid-related disease.

In another aspect, the invention relates to a method of obtaining an antibody that specifically interacts with an amyloid peptide which comprises administering to a non-human mammal a composition comprising a peptide having a sequence as defined in SEQ ID NO:3.

In another aspect, the invention relates to an antibody obtained by a method as defined above or to an antigen-binding fragment thereof.

In another aspect, the invention relates to an agent selected from the group of
(i) an antibody obtained by immunization against a peptide selected from the group of a peptide having the sequence of SEQ ID NO:1 and fragments thereof or an antigen-binding fragment thereof,
(ii) an antibody as defined above or an antigen-biding fragment thereof and
(iii) a combination of one or more of (i) to (ii)
   for use in the treatment or prevention in a non-human mammal of a disease associated with amyloid deposition.

In yet another aspect, the invention relates to a method for the diagnosis of a disease associated with amyloid deposition in a subject comprising contacting a biological sample from said subject with an antibody or antibody mixture wherein said antibody or antibody-mixture comprises an antibody raised specific for a peptide having the sequence of SEQ ID NO:3 and detecting the formation of immune complexes between said antibody and at least an amyloid-containing component within the sample.

### BRIEF DESCRIPTION OF THE DRAWINGS

With the aim of aiding to the comprehension of the present invention, the following figures are enclosed, showing the results achieved with the sequences and antibodies of the same.

FIG. 1: Graphs showing the anti-Abeta IgG titration of serum samples of rabbits immunized with SEQ ID NO 3 (A) and SEQ ID NO 1 (B). X-axis indicates the serum dilution; OD means Optical Density.

FIG. 2: Graphs showings the titration of purified sera from rabbits immunized with peptide sequences SEQ ID NO 3 (A) or SEQ ID NO 1 (B). X-axis indicates the serum dilution; OD means Optical Density. As control, in (C) appears the titration of serum obtained from rabbits only immunized with the adjuvant and the carrier.

FIG. 3: Image of a Dot blot assay. Dot blot detection of soluble Abeta with both the purified Anti-Abeta28-40 peptide (SEQ ID NO 3) and Anti-Abeta1-40 peptide (SEQ ID NO 1) antibodies. Rabbit pre-immune serum was used as control to determine the specificity of the raised antibodies.

FIG. 4: Image with the immunohistochemical detection of Abeta plaques in the dog brain using both the purified Anti-Abeta 28-40 peptide and Anti-Abeta 1-40 peptide antibodies. a) Immunostaining of Abeta plaques (arrows) detected with anti-Abeta 1-40 purified antibodies. b) Immunohistochemistry with pre-immune serum did not presented Abeta plaque immunodetection. c) Immunostaining of Abeta plaques (arrows) detected with anti-Abeta 28-40 purified antibodies. d) Immunostaining of Abeta plaques detected with commercial anti human Abeta peptide antibody, which presented low Immunoreactivity against canine plaques (arrows).

FIG. 5: Graphs showing the titration of the synthesized and purified monoclonal antibody with peptide sequences SEQ ID NO 3 (A) or SEQ ID NO 1 (B). X-axis indicates the serum dilution; OD means Optical Density.

FIG. 6: Image of a Dot blot assay. Dot blot detection of soluble Abeta 28-40 fragment peptide (SEQ ID NO 3) and soluble Abeta 1-40 peptide (SEQ ID NO 1) with the raised monoclonal anti-Abeta 28-40 antibody. Bovine serum albumin was used as control to determine the specificity of the raised antibody.

FIG. 7: Image with the immunohistochemical detection of Abeta plaques in the dog brain using the synthesized monoclonal Anti-Abeta28-40 peptide antibody. a) Immunostaining of Abeta plaques (arrows) detected with monoclonal anti-Abeta 28-40 purified antibody. b) Immunohistochemistry with no primary antibody did not presented Abeta plaque immunodetection.

FIG. 8: Graphs showing the anti-Abeta IgG titration of serum samples of dogs immunized (dark-bars) with Abeta28-40 peptide (SEQ ID NO 3). Un-immunized dogs (clear-bars) received injection of a mixture of adjuvant and carrier in the same proportion than immunized dogs. The arrows indicate the immunization days. X-axis represents the blood extraction days. OD means Optical Density.

FIG. 9: Graphs showing the improvement of cognitive score on day 71 after immunization in 2 dogs treated with the standard pharmacological preparation of 1/3 SEQ ID NO 3 conjugated with KLH added to 2/3 of SEQ ID NO 1. X-axis represents values of each dog (D1 and D2). Pre means pre-immunization; Post means 71 day post-immunization; Score means Cognitive Dysfunction Score

FIG. 10: Graphs showing the increase of Abeta 1-40/42 soluble peptide blood levels in 2 dogs on day 71 after immunization with the standard pharmacological preparation of 1/3 SEQ ID NO 3 conjugated with KLH added to 2/3 of SEQ ID NO 1. X-axis represents each dog (D1 and D2). Pre means pre-immunization, Post means 71 day post-immunization. pg/ml means concentration of Abeta in picogram of Abeta per millilitre of serum

### DETAILED DESCRIPTION OF PARTICULAR EMBODIMENTS

### Active Immunization Methods of the Invention

It has been surprisingly found that certain sequences derived from canine Abeta protein, in particular, the Abeta 1-40 peptide and fragments thereof, induce immunity in non-human mammals without the drawbacks usually associated to this treatment such as severe anaphylactic shocks or allergic reactions. Moreover, these sequences promote beneficial effects on cognition in the non-human mammals suffering from a disease in which diffuse Abeta plaques depositions is observed in the brain. The canine Abeta 1-40 peptide is considered as less immunogenic than the Abeta 1-42 and, as a result, it has not been considered for use in active vaccination of pet and livestock animals. However, the inventors surprisingly found that these sequences derived from canine Abeta 1-40 peptide, induce a relevant immunogenic response in the treated subjects while presenting the advantage of not inducing an allergic reaction, at the same time they promote beneficial effects on cognition disorders.

This effect is illustrated in example 9 of the present invention, wherein aging dogs suffering from cognitive dysfunction are administered a conjugate comprising the amyloid-derived Abeta28-40. The treatment results not only in the production of serum anti-Aβ 1-40 antibodies but in an improvement of the cognitive function of the dogs while producing an increase in the serum levels of soluble Abeta.

Without wishing to be bound by any theory, it is believed that the peptides of the invention induce an immunogenic response that results in the production of anti-Abeta antibodies which are able to remove some of the amyloid deposits. In dogs suffering from cognitive dysfunction wherein the amyloid deposits occur mainly in the brain, the disruption of the amyloids deposits results in the mobilisation of the released beta peptides thus leading to increased peptide levels in peripheral tissues.

Thus, in a first aspect, the invention relates to an agent for use in the treatment or prevention in a non-human mammal of a disease associated with amyloid deposition wherein said agent comprises a peptide selected from the group of a peptide having a sequence of SEQ ID NO:1 (Aβ1-40) and a functionally equivalent variant thereof.

Alternatively, the invention relates to the use of an agent for the manufacture of a medicament for the treatment or prevention in a non-human mammal of a disease associated with amyloid deposition wherein said agent comprises a peptide selected from the group of a peptide having the sequence of SEQ ID NO:1 (Aβ(1-40) and a functionally equivalent variant thereof.

Alternatively, the invention relates to a method for the treatment or prevention in a non-human mammal of a disease associated with amyloid deposition which comprise the administration to said non-human mammal an agent which comprises a peptide selected from the group of a peptide having the sequence of SEQ ID NO:1 (Aβ(1-40)) and a functionally equivalent variant thereof.

The terms “agent” and “therapeutic composition” are used herein interchangeably and refer to a composition comprising a particular antigen, which upon administration to a subject, induces a specific beneficial immune response against that antigen and against related antigens sharing the same or similar epitopes. The beneficial immune response results in a substantial reduction of the severity or progression of a particular disease in the subject, or in the substantial prevention of signs and symptoms of a particular disease in the subject. In the context of the present invention, the vaccine is intended for the treatment and prevention of diseases characterized by amyloid deposition. Accordingly, related terms such as “vaccination” and “vaccinate” refer to administration of the vaccine or therapeutic composition.

The terms “treatment”, “treating”, “prevention”, “preventing” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a non-human mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

The term “non-human mammal”, as used herein, refers, without limitation to a mammal, selected from the group consisting of dogs, cats, monkeys, bears, rabbits, cows, sheep, pigs, guinea pigs, dolphins and Iberian lynxes. In all these non-human mammals the peptide sequence of the Abeta 1-40 and Abeta 1-42 has a high homology degree, comprised between 80% and 100%.

The term “diseases associated with amyloid-deposition”, as used herein interchangeably with “amyloid disease” or “amyloidosis”, relates to any disorder of protein folding affecting humans and animals in which normally soluble homologous proteins are deposited extracellularly with common elements in organ systems leading to tissue damage and disease. Preferably, the amyloidosis results from deposition of the amyloid precursor protein (APP) or its derived fragments in organs such as brain, heart, blood vessels, kidneys, liver, pancreas and lungs giving rise to different disorders. Amyloidosis can be classified according on whether the deposition occurs in a human or in a non-human mammal such as:
In humans: Alzheimer's disease, Down's syndrome, cerebral amyloid angiopathy and hereditary cerebral hemorrhage with amyloidosis. Additionally, different diseases have been described resulting from the accumulation of amyloid deposits in peripheral tissues such as amyloid angiopathy, primary and secondary systemic amyloidosis.
In non-human mammals: A series of diseases collectively known as “cognitive dysfunction syndrome” (CDS) in and including involutive depression, confusional syndrome and dysthymia. Although this disease is found predominantly in dogs, it also affects other animals such as cats. In dogs, CDS, involutive depression, confusional syndrome and dysthymia, all relate to an age-related deterioration of mental function typified by multiple cognitive impairments that affect an afflicted canine's ability to function normally which includes various behavioral disorders. For example, they may not respond to their name or familiar commands, may get lost or confused even in familiar surroundings, may no longer greet or respond to their owners or visitors, may exhibit diminished daytime activity, may walk in circles, may shun affection, and may lose bladder or bowel control.

The term “dysthymia”, as used herein, relates to a condition which typical clinical signs in animals is the loss of the ability to evaluate the relationship between a length of a passage and your own body. The dysthymic animal tendency to force the way and may well remain trapped during hours moaning. Any attempt to foreign aid may trigger aggressive responses. Other clinical signs of dysthymia are going to wrong side of doors; appearing to forget previously learned tasks getting stuck in corners and behind furniture.

The term “cognitive dysfunction syndrome” (CDS) refers to an age-associated condition characterized by a decline in the cognitive abilities of an animal that cannot be attributed to an unrelated general medical condition such as neoplasia, infection, or organ failure. Symptoms of cognitive dysfunction syndrome include, but are not limited to, altered interaction with family members, changes in sleep-wake cycle, decreased activity level, memory loss and inappropriate elimination.

The term “Involutive depression” refers to a condition that includes depression, lethargy, and symptoms of cognitive dysfunction syndrome.

The term “confusional syndrome” refers to a condition that includes daze, hesitation, wandering aimlessly and symptoms of cognitive dysfunction syndrome.

The term “amyloid-related tissue degeneration” as used herein, refers to a process of degeneration of tissue resulting from deposition of amyloid complexes. Amyloid depositions in animals has been shown to affect a series of tissues such as epithelial cells in the bronchus, bronchial glands, gastric and intestinal mucosa, intrahepatic bile ducts, and pancreatic ducts, exocrine glands, vascular walls or connective tissues of small intestines, vascular walls of the heart, lung, liver and thyroid gland as well as in atrioventricular valves as described by Fukuoka et al. (Amyloid, 2004, 11:173-8) and by Uchida et al. (J. Vet. Med. Sci., 1991, 53:1037-42).

The agents of the invention are not limited to the treatment of amyloidosis caused by deposition of the processed beta-amyloid-monomeric peptides such as Abeta peptide, but include other forms of amyloidoses comprising homologous fibrillar proteins derived from at least one of the following precursor proteins: SAA (Serum—Amyloid-Protein A), AL (k or 1-light chains of Immunoglobulins), AH (gl Ig-heavy chains), ATTR (Transthyretin, Serum-Prealbumin), AApo-A-1 (Apolipoprotein A1), AApoA2 (Apolipoprotein A2), AGeI (Gelsolin), ACys (Cystatin C), ALys (Lysozyme), AFib (Fibrinogen), Beta-amyloid (Amyloid precursor protein), Beta-amyloid2M (beta2-microglobulin), APrP (Prion protein), ACaI (Procalcitonin), AIAPP (islet amyloid polypeptide); APro (Prolactin), AIns (Insulin); AMed (Lactadherin); Aker (Kerato-epithelin); ALac (Lactoferrin), Abri (AbriPP), ADan (ADanPP); or AANP (Atrial natriuretical peptide), or neurodegenerative diseases characterized by the deposition of abnormally aggregated forms of endogenous proteins including but not limited to beta-amyloid in Alzheimer's disease, Down's syndrome, cerebral amyloid angiopathy, hereditary cerebral hemorrhage with amyloidosis, Dutch type and Icelandic type alpha-synuclein in Parkinson's disease, dementia with lewy body, multiple system atrophy; Prion protein in Creutzfeldt-Jakob disease and related prion diseases, Huntingtin in Huntington's disease, tau or other neurofibrillary tangle-related proteins in tauopathies including progressive supranuclear palsy (PSP), cortico-basal degeneration (CBD), agyrophilic grain disease (AGD), fronto-temporal dementia (FTD), frontotemporal dementia with Parkinsonism (FTDP 17), Pick bodies in Pick's disease, ataxin in Spinocerebellar ataxia, copper/zinc super oxide dismutase in amyotrophic lateral sclerosis and TDP-43 in frontotemporal lobar degeneration and amyotrophic lateral sclerosis.

The term “Aβ(1-40)”, also known as beta-amyloid peptide, A beta or A4 peptide (see U.S. Pat. No. 4,666,829; Glenner et al (1984) Biochem Biophys Res Commun 120, 1131), is used herein interchangeably with SEQ ID NO:1, and refers to a peptide of 40 amino acids, which is one of the principal component of characteristic plaques of Alzheimer's disease and is generated by processing of the larger protein amyloid precursor protein (APP) by two enzymes, termed beta- and gamma-secretases (Hardy, 1997, Trends Neurosci. 20, 154-159) and which has a sequence of DAEFRHDSGYEVHHQKLVFFAEDVGSNKGAIIGLMVGGVV (SEQ ID NO:1).

The term “having the sequence of SEQ ID NO:”, as used herein, refers to peptides which sequence is defined by the sequence depicted in the respective sequence identifier. Therefore, the term “having” is to be construed as “consisting” and not as “comprising”, thus excluding that the peptides defined by reference to the SEQ ID number actually encompass larger peptides wherein the sequence given by the SEQ ID is a subsequence.

The term “functionally equivalent variant thereof” when referring to the Aβ(1-40) peptide relates to any peptide derived from the Aβ(1-40) by deletion and substitution of one or more of the amino acids in Aβ(1-40) and which maintains substantially the same function as the complete Aβ(1-40) of inducing an immune response when administered to an animal. The variants do not include Aβ(1-42). Moreover, the variants have a sequence length of less than 42 amino acids.

The characterisation of the immunogenic activity of the functionally equivalent variants of the Aβ(1-40) can be determined using the methods described in examples 2 and 6 of the present invention to generate, respectively, polyclonal and monoclonal antibodies and using the methods described in examples 3, 4, 7 and 8 to test the titre and the ability of the antibodies to stain purified Aβ(1-40) spotted on a membrane or amyloid plaques in brain sections from animals suffering from amyloidosis. Moreover, the immunogenic activity of the variants of the invention can be determined by measuring the ability of the antibodies to improve the cognitive defects in aged dogs suffering CDS using the methods as described in example 10 of the invention.

Functional equivalent variants also encompass salts thereof and peptides containing non-natural amino acid derivatives or nonprotein side chains. The term derivative includes any chemical derivative of the peptides of the invention having one or more residues chemically derivatized by reaction of side chains or functional groups. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues such as 2-Aminoadipic acid, N-Ethylasparagine, 3-Aminoadipic acid, Hydroxylysine, beta-alanine, beta-Amino-propionic acid, allo-Hydroxylysine, 2-Aminobutyric acid, 3-Hydroxyproline, 4-Aminobutyric acid, piperidinic, 4-Hydroxyproline acid, 6-Aminocaproic acid, Isodesmosine, 2-Aminoheptanoic acid, allo-Isoleucine, 2-Aminoisobutyricacid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norieucine, 2,3-Diaminopropionic acid, Ornithine and/or N-Ethylglycine. For example: 4-hydroxyproline may be substituted for proline; 5-hydroxylysine may be substituted for lysine; 3-methylhistidine may be substituted for histidine; homoserine may be substituted or serine; and ornithine may be substituted for lysine.

Species analogs may also be termed homologous. Analogs typically differ from naturally occurring peptides at one or a few positions, often by virtue of conservative substitutions. With respect to Abeta peptides, “analogs” typically exhibit at least 80 or 90 percent sequence identity with natural Abeta peptides.

In a preferred embodiment, the functional equivalent of the Aβ(1-40) is a fragment of Aβ(1-40). Suitable fragments of Aβ(1-40) for use in the present invention include any peptide resulting from the deletion of one or more amino acid from the N- or the C-terminus of Aβ(1-40) and include peptides lacking 1, 2, 3, 4, 5, 6, 7 8, 9, 10, 11, 12, 13, 14, 15, 16, 17, 18, 19, 20, 21, 22, 23, 24, 25, 26, 27, 28, 29, 30, 31, 32, 33, 34, 34, 35, 36, 37 or 38 from the N-terminus of Aβ(1-40) or peptides lacking 1, 2, 3, 4, 5, 6, 7 8, 9, 110, 11, 12, 13, 14, 15, 16, 17, 18, 19, 20, 21, 22, 23, 24, 25, 26, 27, 28, 29, 30, 31, 32, 33, 34, 34, 35, 36, 37 or 38 from the C-terminus of Aβ(1-40). Preferred fragments of Aβ(1-40) are depicted in table I.

| Peptide | | SEQ ID NO: |
|---|---|---|
| GLMVGGVV | Aβ(33-40) | 2 |
| KGAIIGLMVGGVV | Aβ(28-40) | 3 |
| IGLMVGGVV | Aβ(32-40) | 4 |
| IIGLMVGGVV | Aβ(31-40) | 5 |
| AIIGLMVGGVV | Aβ(30-40) | 6 |
| GAIIGLMVGGVV | Aβ(29-40) | 7 |
| NKGAIIGLMVGGVV | Aβ(27-40) | 8 |
| SNKGAIIGLMVGGVV | Aβ(26-40) | 9 |

In a more preferred embodiment, the fragment of Aβ(1-40) is the peptide defined by SEQ ID NO:3 corresponding to Aβ(28-40) and having a peptide sequence KGAIIGLMVGGVV. In a still more preferred embodiment, the agent is the peptide defined by SEQ ID NO:1 and corresponding to Aβ(1-40). In a still more preferred embodiment, the agent is a combination of the Aβ(28-40) and the Aβ(1-40) peptides. The two peptides may be combined at molar or weight ratios of 1:1000 to 1000:1, preferably 1:99 to 99:1, preferably 10:90 to 90:10, more preferably 20:80 to 80:20, more preferably 30:70 to 70:30, more preferably 40:60 to 60:40.

In a preferred embodiment, the functionally equivalent variants of the Aβ(1-40) peptide are those which lack substantially the ability to aggregate or to form higher order structures such as fibrils, glomerules, oligomers or amyloid plaques. Methods suitable for the determination of the ability of an Aβ(1-40) to form fibrils are known to the skilled person and include methods based on the detection of fibrils using Congo red staining as described by Wood et al. (J. Biol. Chem., 1996, 271:4086-4092) or thioflavin T as described by LeVine (Protein Sci, 1993, 2:404-410), by immunological assays as described by Howlett et al. (Biochem J., 1999, 340:283-289) or by direct visualization using electron microscope of the uranyl acetate fibrils. The expression “lacking substantially the ability of forming higher order structures” relates to variants of the Aβ(1-40) wherein the ability of forming fibrils has been reduced by at least 50%, by at least 60%, by at least 70%, by at least 80%, by at least 90%, by at least 95% or by 100%, i.e. it has been completely abrogated.

Proteins or peptides suitable for use in the present invention may be made by any technique known to those of skill in the art, including the expression of proteins, polypeptides or peptides through standard molecular biological techniques, or the chemical synthesis of proteins or peptides. Alternatively, various commercial preparations of proteins, polypeptides and peptides are known to those of skill in the art and proteins or peptides of specific sequence may be obtained from a variety of commercial vendors known in the art. Peptides containing derivatized amino acid residues may be prepared by incorporating such residues into the peptide chain during synthesis. Alternatively, modified amino acid residues may be prepared by chemical derivatization after peptide synthesis, for example using side-chain specific chemical modifying agents well known in the art. Another embodiment for the preparation of polypeptides according to the invention is the use of peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used to engineer second generation molecules having many of the natural properties of the peptides of the invention (=binding peptides) disclosed herein, but with altered and even improved characteristics.

Further, a veterinary vaccine composition can include an immunogenic polypeptide chain formed by two or more molecules of any fragment of Abeta(1-40) as defined above wherein said two or more molecules form a single polypeptide chain. In this case, the different immunogenic peptides may be directly linked or may be separated by one or more amino acids used as spacers.

In a preferred embodiment, the agent of the invention is used as a covalent conjugate with an immunogenic carrier. Suitable carriers include serum albumins, keyhole limphet hemocyanin, immunoglobulin molecules, thyroglobulin, ovalbumin, Neisseria meningitidies outer membrane protein complex (OMPC), tetanus toxoid, or a toxoid from other pathogenic bacteria, such as diphtheria, E. coli, cholera, or H. pylori, or an attenuated toxin derivative. Other carriers include T-cell epitopes that bind to multiple MHC alleles, e.g., at least 75 percent of all human MHC alleles. Such carriers are sometimes known in the art as “universal T-cell epitopes.” Examples of universal T-cell epitopes include Influenza Hemagluttinin: HA₃₀₇₋₃₁₉, PADRE, Malaria CS: T3 epitope, Hepatitis B surface antigen: HBsAg₁₉₋₂₈, Heat Shock Protein 65: hsp65₁₅₃₋₁₇₁, bacille Calinette-Guerin, Tetanus toxoid: TT₈₃₀₋₈₄₄ Tetanus anus toxoid: TI₉₄₇₋₉₆₇, HIV gp120 TI. Immunogenic agents can also be linked to peptides that enhance transport across tissues, as described in WO 97/17613 and WO 97/17614. In a preferred embodiment, the carrier is keyhole limpet hemocyanine.

Immunogenic agents can be linked to carriers by chemical crosslinking. Techniques for linking an immunogen to a carrier include the formation of disulfide linkages using N-succinimidyl-3-(2-pyridyl-thio) propionate (SPDP) and succinimidyl 4-(N-maleimidomethyl)cyclohexane-1-carboxylate (SMCC) (if the peptide lacks a sulfhydryl group, this can be provided by addition of a cysteine residue). These reagents create a disulfide linkage between themselves and peptide cysteine residues on one protein, and an amide linkage through the .epsilon.-amino on a lysine, or other free amino group in other amino acids. Other bifunctional coupling agents form a thioether rather than a disulfide linkage. Many of these thio-ether-forming agents are commercially available and include reactive esters of 6-maleimidocaproic acid, 2-bromoacetic acid, and 2-iodoacetic acid, 4-(N-maleimido-methyl)cyclohexane-1-carboxylic acid. The carboxyl groups can be activated by combining them with succinimide or 1-hydroxyl-2-nitro-4-sulfonic acid, sodium salt. Immunogenic peptides can also be expressed as fusion proteins with carriers. The immunogenic peptide can be linked at the amino terminus, the carboxyl terminus, or internally to the carrier. Optionally, multiple repeats of the immunogenic peptide can be present in the fusion protein.

Coupling of the immunogenic agent to the carrier protein can be carried out under conditions adequate for achieving a desired ratio of molecules of immunogenic agent per molecule of carrier protein. Thus, depending on the molar ratio of the carrier protein and the immunogenic protein, immunogenic agent/carrier ratios of 1:1, 2:1, 4:1, 5:1, 10:1, 20:1, 50:1, 100:1 and more can be achieved.

The authors of the present invention have also observed that a conjugate comprising the Aβ(28-40) peptide and an immunologically active carrier wherein the peptide and the carrier are covalently coupled is particularly adequate for the treatment of cognitive dysfunctions in dogs as observed by the antibody response obtained in these animals (see example 9 of the present invention) and by the improvement in the cognitive functions (see example 10 of the present invention). Thus, in another aspect, the invention relates to a conjugate comprising a peptide consisting of Aβ(28-40) and an immunologically active carrier wherein the peptide and the carrier are covalently coupled.

Preferred carriers and means for conjugation have been described above and are equally applicable to the conjugate as defined herein.

Moreover, the present invention also comprises compositions which comprise the peptide consisting of Aβ(28-40) and one or more additional immunogenic components. Thus, in another aspect, the invention relates to a composition comprising a first component and a second component selected from the group of
(i) a composition wherein the first component comprises a peptide having a sequence of SEQ ID NO:3 and the second component comprises one or more peptides selected from the group of a peptide having a sequence of SEQ ID NO:10 and a functionally equivalent variant thereof,
(ii) a composition wherein the first component comprises a peptide having a sequence of SEQ ID NO:3 and the second component comprises one or more peptides selected from the group of a peptide having a sequence of SEQ ID NO:10 and a functionally equivalent variant thereof and wherein the peptide forming part of the first component and/or the peptide or peptides forming part of the second component are covalently coupled to an immunologically active carrier and
(iii) a composition comprising a first component comprising a peptide having a sequence of SEQ ID NO:3 and a second component comprising one or more peptides selected from the group of a peptide having the sequence of SEQ ID NO:10 and a functionally equivalent thereof wherein both the peptide forming the first component and the peptide or peptides forming the second component are covalently coupled to a single immunologically active carrier.

The term “composition”, as used herein, refers to a mixture of two or more bio-active agents and defines especially a “kit of parts” in the sense that the different components can be dosed independently or by use of different fixed combinations with distinguished amounts of these components i.e., simultaneously or at different time points. Moreover, these terms comprise a commercial package comprising (especially combining) as active ingredients the components of the compositions, together with instructions for simultaneous, sequential (chronically staggered, in time-specific sequence, preferentially) or (less preferably) separate use thereof in the delay of progression or treatment of a proliferative disease. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (as can be determined according to standard methods). The ratio of the total amounts of the components of the composition to be administered in the combined preparation can be varied, e.g., in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners, in particular a more than additive effect, which hence could be achieved with lower doses of each of the combined drugs, respectively, than tolerable in the case of treatment with the individual drugs only without combination, producing additional advantageous effects, e.g., less side effects or a combined therapeutic effect in a non-effective dosage of one or both of the combination partners (components), and very preferably a strong synergism of the combination partners (a) and (b).

The composition can be prepared for any combination of simultaneous, sequential and separate use, meaning that the components may be administered at one time point simultaneously, followed by administration of only one component with lower host toxicity either chronically, e.g., more than 3-4 weeks of daily dosing, at a later time point and subsequently the other component or the combination of both components at a still later time point (in subsequent drug combination treatment courses for an optimal anti-tumor effect) or the like.

The terms SEQ ID NO:3, corresponding to the peptide Aβ(28-40), as been defined above.

“Aβ(1-42)”, also known as beta-amyloid peptide, A beta or A4 peptide (see U.S. Pat. No. 4,666,829; Glenner et al (1984) Biochem Biophys Res Commun 120, 1131), as used herein, refers to a peptide of 42 amino acids, which is one of the principal component of characteristic plaques of Alzheimer's disease and is generated by processing of the larger protein amyloid precursor protein (APP) by two enzymes, termed beta- and gamma-secretases (Hardy, 1997, Trends Neurosci. 20, 154-159) and which has a sequence of DAEFRHDSGYEVHHQKLVFFAEDVGSNKGAIIGLMVGGVVIA (SEQ ID NO:10).

The term “functionally equivalent variant thereof” when referring to the Aβ(1-42) peptide relates to any peptide derived from the Aβ(1-42) by deletion and substitution of one or more of the amino acids in Aβ(1-42) and which maintains substantially the same function as the complete Aβ(1-42) of inducing an immune response when administered to an animal.

The characterisation of the immunogenic activity of the functionally equivalent variants of Aβ(1-42) can be carried out essentially as defined above in respect of the functionally equivalent variants of Aβ(1-40).

In a first type of compositions according to the present invention, the first component is the peptide Aβ(28-40) and the second and successive components are the Aβ(1-42) peptide or one or more functional equivalents variants thereof. Suitable functional equivalents variants for use in the compositions include fragments. Suitable fragments of Aβ(1-42) include the Aβ(1-40) as well as any of the fragments of Aβ(1-40) defined above. In a preferred embodiment, the composition comprises, in addition to the Aβ(28-40) peptide, a single fragment of Aβ(1-42). In a still more preferred embodiment, the single fragment of Aβ(1-42) is Aβ(1-40).

In a second type of compositions according to the present invention, the first component (the peptide Aβ(28-40)) and/or the second and successive component(s) (Aβ(1-42) and/or a functionally equivalent variant thereof) may be covalently coupled to an immunologically active carrier. As defined above, suitable functionally equivalent variant for use in the compositions include any fragment of Aβ(1-42) and, in particular, the Aβ(1-40) as well as any of the fragments of Aβ(1-40) defined above. In a preferred embodiment, the composition comprises, in addition to the Aβ(28-40) peptide, a single fragment of Aβ(1-42). As the skilled person will appreciate, the compositions may comprise a first component consisting of a conjugate of Aβ(28-40) and a carrier and a second component consisting non-conjugated Aβ(1-42) and/or one or more fragments thereof. Alternatively, the composition may comprise a first component consisting of non-conjugated Aβ(28-40) and a second component consisting of conjugated Aβ(1-42) and one or more conjugated fragments thereof. Alternatively, both the first and second component may be provided as conjugates. Suitable carriers for use in the conjugates forming the compositions of the invention are essentially as described above. In a preferred embodiment, the composition comprises a conjugate of Aβ(28-40) and non-conjugated Aβ(1-40). In a still more preferred embodiment, the Aβ(28-40) peptide is conjugated to KLH. In a still more preferred embodiment, the Aβ(28-40)-KLH conjugate forms one third of the total amount of active component and the Aβ(1-40) peptide forms two thirds of the active component.

In a third type of composition, the peptide consisting of Aβ(28-40) and the one or more peptides selected from the group of Aβ(1-42) and/or fragments thereof are covalently coupled to a single immunologically active carrier. In a preferred embodiment, the single carrier is keyhole limpet hemocyanin. For this type of composition, the coupling reaction is carried out using a single carrier molecule and the two or more immunogenic agents so that the carrier is simultaneously modified by the different immunogenic agents.

As shown in the examples of the present invention, the conjugates and compositions of the present invention are capable of inducing an immune response in different model animals as well as of inducing an improvement in the cognitive capacities of aging dogs suffering from CDS. Accordingly, in another aspect, the invention provides conjugates and compositions of the invention for use in medicine. For this purpose, the compositions and conjugates are ideally formulated as pharmaceutical compositions by combining with a pharmaceutically acceptable carrier. Thus, in another aspect, the invention provides a pharmaceutical composition comprising any of the agents or compositions of the invention and a pharmaceutically acceptable carrier.

In another aspect, the invention relates to a conjugate of the invention as defined above or to a composition of the invention as defined above for use in the treatment of an amyloid-related disease.

Alternatively, the invention relates to the use of a conjugate of the invention as defined above or of a composition of the invention as defined above for the manufacture of a medicament for the treatment of an amyloid-related disease.

Alternatively, the invention relates to a method of treatment of an amyloid-related disease in a subject comprising the administration to said subject a conjugate of the invention as defined above or of a composition of the invention as defined above.

The skilled person will appreciate that the conjugates and agents of the invention are adequate for the treatment of veterinary diseases associated with amyloid deposition such as involutive depression, confusional syndrome, dysthymia and cognitive dysfunction syndrome (CDS) and amyloid-related tissue degeneration.

However, the dog has been considered as a suitable model for human diseases associated with amyloid deposition and thus, the improvements observed in the animal models indicate that the conjugates are equally suitable for the treatment of diseases related with amyloid depositions in human patients such as localised amyloidosis, systemic amyloidosis, secondary amyloidosis, scrapie, bovine spongiform encephalitis, Creutzfeld-Jacob disease, Alzheimer's disease, cerebral amyloid angiopathy.

The agents and compositions of the invention may be administered together with a pharmaceutically-accepted adjuvant. The adjuvant increases the titer of induced antibodies and/or the binding affinity of induced antibodies relative to the situation if the agent or composition were used alone.

Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPL™) (see GB2220211 (RIBI ImmunoChem Research Inc., Hamilton, Mont., now part of Corixa), RC-529 (Corixa, Hamilton, Mont.). STIMULON™ QS-21 is a triterpene glycoside or saponin isolated from the bark of the Quillaja Saponaria Molina tree found in South America (see Kensi I et al, in Vaccine Design: The Subunit and Adjuvant Approach (eds. Powell and Newman, Plenum Press, NY, 1995); U.S. Pat. No. 5,057,540), (Aquila BioPharmaceuticals, Framingham, Mass.). Other adjuvants are oil in water emulsions (such as squalene or peanut oil), optionally in combination with immune stimulants, such as monophosphoryl lipid A (see Stoute et al, N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

A preferred class of adjuvants is aluminum salts (alum), such as alum hydroxide, alum phosphate, alum sulfate. Such adjuvants can be used with or without other specific immunostimulating agents such as MPL or 3-DMP, QS-21, polymeric or monomeric amino acids such as polyglutamic acid or polylysine. Another class of adjuvants is oil-in-water emulsion formulations. Such adjuvants can be used with or without other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(r-2′ dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) THERAMIDE™), or other bacterial cell wall components. Oil-in-water emulsions include (a) MF59 (WO 90/14837), containing 5 percent Squalene, 0.5 percent Tween 80, and 0.5 percent Span 85 (optionally containing various amounts of MTP-PE) formulated into submicron particles using a micro fluidizer such as Model HOY microfluidizer (Microfluidics, Newton Mass.), (b) SAF, containing 10 percent Squalene, 0.4 percent Tween 80, 5 percent pluronic-blocked polymer L121, and thr-MDP, either microfluidized into a submicron emulsion or vortexed to generate a larger particle size emulsion, and (c) RIB I™ adjuvant system (RAS), (Ribi ImmunoChem, Hamilton, Mont.) containing 2 percent squalene, 0.2 percent Tween 80, and one or more bacterial cell wall components from the group consisting of monophosphoryllipid A (MPL), trehalose dimycolate (TDM), and cell wall skeleton (CWS), preferably MPL+CWS (DETOX™).

Another class of preferred adjuvants is saponin adjuvants, such as STIMULON™ (QS-21, Aquila, Framingham, Mass.) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-I a and beta peptides, IL-2, IL-4, IL-6, IL-12, IL 13, and IL-15), macrophage colony stimulating factor (M-CSF), granulocyte-macrophage colony stimulating factor (GM-CSF), tumor necrosis factor (TNF), chemokines, such as MIP la and beta and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

An adjuvant can be administered with an immunogen as a single composition, or can be administered before, concurrent with, or after administration of the immunogen. Immunogen and adjuvant can be packaged and supplied in the same vial or can be packaged in separate vials and mixed before use. Immunogen and adjuvant are typically packaged with a label indicating the intended therapeutic application. If immunogen and adjuvant are packaged separately, the packaging typically includes instructions for mixing before use. The choice of an adjuvant and/or carrier depends on the stability of the immunogenic formulation containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, Complete Freund's adjuvant is not suitable for human administration. Alum, MPL and QS-21 are preferred. Optionally, two or more different adjuvants can be used simultaneously. Preferred combinations include alum with MPL, alum with QS-21, MPL with QS-21, MPL or RC-529 with GM-CSF, and alum, QS-21 and MPL together. Also, Incomplete Freund's adjuvant can be used (Chang et ah, Advanced Drug Delivery Reviews 32, 173-186 (1998)), optionally in combination with any of alum, QS-21, and MPL and all combinations thereof.

Agents for the treatment of amyloidosis can be administered by parenteral, topical, intravenous, oral, subcutaneous, intra-arterial, intracranial, intraperitoneal, intranasal or intramuscular means for prophylactic and/or therapeutic treatment. The most typical route of administration of an immunogenic agent is subcutaneous although other routes can be equally effective. The next most common route is intramuscular injection. This type of injection is most typically performed in the arm or leg muscles. In some methods, agents are injected directly into a particular tissue where deposits have accumulated, e.g., intracranial injection. Intramuscular injection or intravenous infusion is preferred for administration of antibody (in combination therapies). In some methods, particular therapeutic antibodies are injected directly into the cranium. In some methods, antibodies are administered as a sustained release composition or device, such as a MEDIP AD™ device.

Agents of the invention are often administered as pharmaceutical compositions comprising an active therapeutic agent, i.e., and a variety of other pharmaceutically acceptable components. See Remington's Pharmaceutical Science (15th ed., Mack Publishing Company, Easton, Pa., 1980). The preferred form depends on the intended mode of administration and therapeutic application. The pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpynolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20 and polysorbate 80, Triton, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, REMINGTON'S PHARMACEUTICAL SCIENCES, 18^{th} Edition, (A. R. Gennaro, ed.), 1990, Mack Publishing Company.

Effective doses of the therapeutic compositions and agents of the present invention, vary depending upon many different factors, including means of administration, target site, physiological state of the mammal, whether the patient is a human or an animal, other medications administered, and whether treatment is prophylactic or therapeutic. Treatment dosages need to be titrated to optimize safety and efficacy. The amount of immunogen depends on whether adjuvant is also administered, with higher dosages being required in the absence of adjuvant. The amount of an immunogen for administration sometimes varies from 0.1-500 μg per mammal and more usually from 5-500 μg per injection for human administration. Occasionally, a higher dose of 1-2 mg per injection is used. Typically about 10, 20, 50 or 100 μg is used for each human injection. The timing of injections can vary significantly from once a day, to once a year, to once a decade. On any given day that a dosage of immunogen is given, the dosage is greater than 1 microgram/patient and usually greater than 10 μg/patient if adjuvant is also administered. In the absence of adjuvant, the dosage is greater than 10 μg/patient and usually greater than 100 microgram/patient. A typical regimen consists of an immunization followed by booster injections at 6 weekly intervals. Another regimen consists of an immunization followed by booster injections 1, 2 and 12 months later. Another regimen consists of an injection every two months for life. Alternatively, booster injections can be on an irregular basis as indicated by monitoring of immune response.

Agents for inducing an immune response can be administered by parenteral, topical, intravenous, oral, subcutaneous, intraperitoneal, intranasal, or intramuscular means for prophylactic and/or therapeutic treatment. The most typical route of administration is subcutaneous, although others can be equally effective. The next most common is intramuscular injection. This type of injection is most typically performed in the arm or leg muscles. Intravenous injections as well as intraperitoneal injections, intraarterial, intracranial, or intradermal injections are also effective in generating an immune response. In some methods, agents are injected directly into a particular tissue where deposits are accumulated. Intranasal immunization was successfully used to increase the production of anti-A beta antibodies in wildtype mice (Lemere et al. 2000, Ann. N.Y. Acad. Sci. 920:328-331). Vaccine or therapeutic compositions of the invention can optionally comprise other agents that are at least partly effective in treatment of diseases associated with beta-amyloid formation and/or aggregation. In the case of Alzheimer's and Down's syndrome, in which beta-amyloid aggregation occurs in the brain, the vaccine or therapeutic composition of the invention may also comprise other agents that increase passage of the active components of the composition of the invention across the blood-brain barrier.

The immune response to the vaccine composition can be improved by delivery through entrapment in or on biodegradable microparticles. The immunogenic agent can be encapsulated with or without an adjuvant, in or on biodegradable microparticles, to potentiate immune responses and to provide time-controlled release for sustained or periodic responses.

The veterinary and pharmaceutical compositions of the present invention can be complemented with any other agent which is commonly used for treating these kinds of diseases. For instance, the treatment of Alzheimer's disease may be carried out using a combination of one of the agents and composition of the invention and therapy which includes, but is not limited to one or more of the following: antioxidants (e.g., alpha-tocopherol); cholinesterase inhibitors (e.g., tacrine, donepezil, rivastigmine, galantamine, and metrifonate); N-Methyl-D-Aspartate (NMDA) antagonists (e.g., memantine, amantadine, rimantadine, and ketamine); anti-inflammatory agents (e.g., propentifyline and selective COX2 inhibitors, for example, celecoxib and rofecoxib); chelating agents (e.g., cliquinol); oestrogens (e.g., selective oestrogen receptor modulators); or secretase inhibitors.

### Passive Immunization Methods of the Invention

The invention is based, at least in part, on the identification and characterization of antibodies that specifically bind to amyloid fibrillar/non-fibrillar polypeptides and are effective at reducing plaque burden and/or reducing the neuritic dystrophy associated with amyloidogenic disorders. Structural and functional analysis of these antibodies leads to the design of various humanized antibodies for prophylactic and/or therapeutic use.

Thus, in another aspect, the invention relates to a method of obtaining an antibody that specifically recognises an amyloid peptide which comprises administering to a non-human mammal a composition comprising a peptide having the sequence of SEQ ID NO:3 (Aβ(28-40)).

The term “specific recognition”, “specifically recognizing”, “specifically binding with”, “specifically reacting with” or “specifically forming an immunological reaction with” refers to a binding reaction by the antibody to the beta-amyloid or to amyloid deposits containing said beta amyloid peptide, which is determinative of the presence of the beta-amyloid in the sample tested, in the presence of a heterogeneous population of other proteins.

Various host animals can be immunized for injection with the Aβ(28-40) peptide according to the invention, including, but not limited to, mouse, rat, rabbit, chicken, camelid or sheep or may be a transgenic version of any of the animals mentioned above, for example a transgenic mouse with human immunoglobulin genes, which produces human antibodies after an antigenic stimulus. Other types of animals which may be immunized include mice with severe combined immunodeficiency (SCID) which have been reconstituted with human peripheral mononuclear blood cells (chimeric hu-PBMC SCID mice) or with lymphoid cells or precursors thereof, as well as mice which have been treated with a lethal total body irradiation, then protected against radiation with bone marrow cells from a mouse with severe combined immunodeficiency (SCID) and subsequently transplanted with functional human lymphocytes (the “Trimera” system). Another type of an animal to be immunized is an animal (e.g. a mouse) in whose genome an endogenous gene encoding the antigen of interest has been switched off (knocked out), for example by homologous recombination, so that, after immunization with the antigen, said animal recognizes said antigen as foreign. It is obvious to the skilled worker that the polyclonal or monoclonal antibodies produced by this method are characterized and selected by using known screening methods which include, but are not limited to, ELISA techniques.

If the peptides of the invention are not or only weakly immunogenic, their immunogenicity may be increased by coupling them to carriers, preferably to a carrier protein as defined above using commonly known possible couplings available to the skilled worker.

Various adjuvants may be used to enhance the immunological response, depending on the host species, including, but not limited to, complete or incomplete Freund's adjuvant, a mineral gel such as aluminum hydroxide, surface active substances such as lysolecithin, pluronic polyol, a polyanion, a peptide, an oil emulsion, keyhole limpet hemocyanin, dinitrophenol, or an adjuvant such as BCG (bacille Calmette-Guerin) or Corynebacterium parvum.

For the preparation of monoclonal antibodies, any technique which provides for the production of antibody molecules by continuous cell lines in culture may be used. Hyperimmunization of an appropriate donor, generally a mouse, with the antigen is undertaken. Isolation of splenic antibody producing cells is then carried out. These cells are fused to a cell characterized by immortality, such as a myeloma cell, to provide a fused cell hybrid (Hybridoma) which can be maintained in culture and which secretes the required monoclonal antibody. The cells are then cultured in bulk and the monoclonal antibodies harvested from the culture media for use. Specific techniques include but are not limited to the hybridoma technique developed by Kohler and Milstein (1975), the human B-cell hybridoma technique (Kozbor et al., 1983) or the EBV-hybridoma technique to produce human monoclonal antibodies (Cole et al, 1985). Screening for the desired antibody can be done by techniques known in the art, such as ELISA. For selection of an antibody that specifically binds the amyloid beta peptide but that does not specifically bind another protein can be done on the basis of positive binding to the first and the lack of binding to the second. Thus, in a particular embodiment, the present invention provides an antibody that binds with greater affinity (particularly at least 2-fold, more particularly at least 5-fold, still more particularly at least 10-fold greater affinity) to an N-terminal truncated and/or post-translationally A beta peptide than to another protein.

For the preparation of polyclonal antibodies, the selected animal host is injected with the immunization cocktails, preferably subcutaneously. The antibody titers may be determined using an immunoassay, for example competitively using a sheep antiserum directed against host IgG and a labeled amyloid peptide. Thus it may be decided toward the end of immunization whether a particular host is suitable for producing antibodies. If, for example, four immunizations are carried out, it is possible to determine the antibody titer after the third immunization and then to obtain antibodies from animals having a sufficient antibody titer. The antibodies produced are preferably obtained by taking blood from the hosts over a period of several weeks or months. Finally, the host can be bled. Serum containing the desired antibodies may be obtained from the blood obtained in a manner known per se. The whole serum thus obtained may, if required, be further purified by the skilled worker in order to concentrate the antibody fraction present therein and in particular the oligomer-recognizing antibodies.

Antibodies that recognize the amyloid peptide can be identified in a simple immunoassay showing the ability of one antibody to block the binding of another antibody to a target antigen, i.e., a competitive binding assay. Competitive binding is determined in an assay in which the immunoglobulin under test inhibits specific binding of a reference antibody to a common antigen, such as Abeta. Numerous types of competitive binding assays are known, for example: solid phase direct or indirect radioimmunoassay (RIA), solid phase direct or indirect enzyme immunoassay (ETA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more.

In a preferred embodiment, the antibodies are affinity purified using methods commonly known to the skilled person. For affinity purification of polyclonal or monoclonal anti-amyloid peptide antibodies a suitable amyloid peptide column has to be prepared. Purified amyloid peptide or a conjugate thereof is fixed by a standard protocol to a suitable solid supports as for example are Sepharose or Affi-gel, activated to covalently the antigen to the support (suitable activated solid supports are for example available from Pierce, Rockford, USA). An affinity column is then prepared from said antigen-carrying resin.

Successful affinity purification of antibody depends on effective presentation of the relevant epitopes on the antigen to binding sites of the antibody. If the antigen is small and immobilized directly to a solid support surface by multiple chemical bonds, important epitopes may be blocked or sterically hindered, prohibiting effective antibody binding. Therefore, it is best to immobilize antigens using a unique functional group (e.g., sulfhydryl on a single terminal cysteine in a peptide) and to use an activated support whose reactive groups occur on spacer arms that are several atoms long. For larger antigens, especially those with multiple sites of immobilization, the spacer arm length becomes less important since the antigen itself serves as an effective spacer between the support matrix and the epitope.

Little variation normally exists among typical binding and elution conditions for affinity purification of antibodies because at the core of each procedure is the affinity of an antibody for its respective antigen. Since antibodies are designed to recognize and bind antigens tightly under physiologic conditions, most affinity purification procedures use binding conditions that mimic physiologic pH and ionic strength. The most common binding buffers are phosphate buffered saline (PBS) and Tris buffered saline (TBS) at pH 7.2 and 1.5 M NaCl (premixed buffer packs are for example available from Pierce, Rockford, USA). Once the antibody has been bound to an immobilized antigen, additional binding buffer is used to wash unbound material from the support. To minimize non-specific binding, the wash buffer may contain additional salt or detergent to disrupt any weak interactions.

Specific, purified antibodies are eluted from an affinity resin by altering the pH and/or ionic strength of the buffer (common elution buffers are for example available from Pierce, Rockford, USA). Antibodies in general are resilient proteins that tolerate a range of pH from 2.5 to 1 1.5 with minimal loss of activity, and this is by far the most common elution strategy. In some cases an antibody-antigen interaction is not efficiently disrupted by pH changes or is damaged by the pH, requiring that an alternate strategy be employed.

In another aspect, the invention relates to an antibody obtained using the methods as defined above or an antigen-binding fragment thereof.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as the myriad imnunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. The basic immunoglobulin (antibody) structural unit is known to comprise a tetramer or dimer. Each tetramer is composed of two identical pairs of polypeptide chains, each pair having one “light” (about 25 kD) and one ‘heavy” chain (about 50-70 kD). The N-terminus of each chain defines a variable region of about 100 to 110 or more amino acids, primarily responsible for antigen recognition. The terms “variable light chain (V_{L})” and “variable heavy chain (V_{H})” refer to these variable regions of the light and heavy chains respectively. Optionally, the antibody or the immunological portion of the antibody, can be chemically conjugated to, or expressed as, a fusion protein with other proteins.

The term “antigen-binding fragment” of an antibody (or simply “antibody portion,” or “fragment”), as used herein, refers to one or more fragments of a full-length antibody that retain the ability to specifically bind to the antigen. These fragments include, without limitation: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al Such single chain antibodies are also encompassed within the term “antigen-binding fragment” of an antibody

Antibodies of the invention include, but are not limited to polyclonal, monoclonal, bispecific, human, humanized or chimeric antibodies, single variable fragments (ssFv), single chain fragments (scFv), Fab fragments, F(ab′) fragments, fragments produced by a Fab expression library, anti-idiotypic antibodies and epitope-binding fragments of any of the above, provided that they retain the original binding properties. Also mini-antibodies and multivalent antibodies such as diabodies, triabodies, tetravalent antibodies and pentabodies can be used in a method of the invention. The preparation and use of these fragments and multivalent antibodies has been described extensively in International Patent Application WO 98/29442. The immunoglobulin molecules of the invention can be of any class (i.e. IgG, IgE, IgM, IgD and IgA) or subclass of immunoglobulin molecules.

While various antibody fragments are defined in terms of enzymatic digestion of an intact antibody with papain, pepsin or other proteases, one of skill will appreciate that such antibody fragments as well as full size antibodies may be synthesized de novo either chemically or by utilizing recombinant DNA methodology. Thus, the term antibody, as used herein, also includes antibodies and antibody fragments either produced by the modification of whole antibodies or synthesized de novo using recombinant DNA methodologies.

Heteroconjugate antibodies, comprising two covalently joined antibodies, are also within the scope of the invention. Heteroconjugate antibodies may be made using any convenient cross-linking methods. Suitable cross-linking agents and techniques are well known in the art, and are described in U.S. Pat. No. 4,676,980.

To improve the nature of the antibodies of the invention or fragments thereof, these can be modified by making them less immunogenic, for example by adapting them to the recipient specie. This is done with standard procedures, using as the constant part of the antibodies those parts of the recipient specie attached to the variable region of the antibodies according to the invention or any of its fragments.

In another aspect, the invention relates to an agent selected from the group of
(i) an antibody obtained by immunization against a peptide selected from the group of a peptide having the sequence of SEQ ID NO:1 and fragments thereof or an antigen-binding fragment thereof,
(ii) an antibody as defined above or an antigen-biding fragment thereof and
(iii) a combination of one or more of (i) to (ii)
   for use in the treatment or prevention in a non-human mammal of a disease associated with amyloid deposition.

Alternatively, the invention relates to the use of an agent selected from the group of
(i) an antibody obtained by immunization against a peptide selected from the group of a peptide having the sequence of SEQ ID NO:1 and fragments thereof or an antigen-binding fragment thereof,
(ii) an antibody as defined above or an antigen-biding fragment thereof and
(iii) a combination of one or more of (i) to (ii)
   for the manufacture of a medicament for the treatment or prevention in a non-human mammal of a disease associated with amyloid deposition.

Alternatively, the invention relates to a method for the treatment or prevention in a non-human mammal of a disease associated with amyloid deposition comprising administering to said non-human mammal an agent selected from the group of
(i) an antibody obtained by immunization against a peptide selected from the group of a peptide having the sequence of SEQ ID NO:1 and fragments thereof or an antigen-binding fragment thereof,
(ii) an antibody as defined above or an antigen-biding fragment thereof and
(iii) a combination of one or more of (i) to (ii)

Diseases related to amyloid deposition in non-human mammals that can be treated using the antibodies according to the present invention have been described in detail above and include, without limitation, involutive depression, confusional syndrome, dysthymia, cognitive dysfunction syndrome (CDS) and amyloid-related tissue degeneration.

For passive immunization with an antibody, the dosage ranges from about 0.0001 to 100 mg/kg, and more usually 0.01 to 5 mg/kg of the host body weight. Effective doses of the therapeutic compositions (e.g. antibodies) of the present invention, vary depending upon many different factors, including means of administration, target site, physiological state of the mammal, whether the patient is a human or an animal, other medications administered, and whether treatment is prophylactic or therapeutic. Treatment dosages need to be titrated to optimize safety and efficacy. The amount of peptides/recognition molecules of the invention depends on whether adjuvant is also administered, with higher dosages being required in the absence of adjuvant. The amount of peptides/recognition molecules of the invention for administration sometimes vary from 1-500 microgram per mammal and more usually from 5-500 microgram per injection for human administration. Occasionally, a higher dose of 1-2 mg per injection is used. Typically about 10, 20, 50 or 100 microgram is used for each human injection. The timing of injections can vary significantly from once a day, to once a year, to once a decade. On any given day that a dosage of the peptides/recognition molecules of the invention is given, the dosage is greater than 1 microgram/patient and usually greater than 10 microgram/patient if adjuvant is also administered. In the absence of adjuvant, the dosage is greater than 10 microgram/patient and usually greater than 100 microgram/patient.

A person skilled in the art would select an appropriate administration route for the antibodies of the present invention. Antibodies can be administered via any conventional route such as, subcutaneous, oral, bucal, intraocular, intramuscular, parenteral, enteral, transdermal, depot, intravaginal or rectal administration, or in a form suitable for administration by inhalation or insufflation (either through the mouth, the nose or the ears). The antibodies can be administered in a single dose or in multiple doses. A suitable treatment schedule is readily determined and available to one of ordinary skill in the art.

Various adjuvants may be used to enhance the immunological response of the antibody of the invention, depending on the host species, including, but not limited to, complete or incomplete Freund's adjuvant, a mineral gel such as aluminum hydroxide, surface active substances such as lysolecithin, pluronic polyol, a polyanion, a peptide, an oil emulsion, keyhole limpet hemocyanin, dinitrophenol, or an adjuvant such as BCG (bacille Calmette-Guerin) or Corynebacterium parvum.

Similarly to the active immunization methods, the compositions of the invention for use in the passive immunization methods may be encapsulated or formulated as different sustained-released forms. Moreover, the compositions may also be administered as combined therapy with other therapies known to be useful for the treatment of Alzheimer's disease in particular and amyloidosis in general. Suitable therapies have been described above in the context of the active immunization methods.

### Diagnostic Methods of the Invention

Animals that suffer from a disease which is characterized by amyloid depositions in the brain or other tissues will have soluble Abeta peptide fragments in CSF or blood. Thus, the availability of antibodies specific for the Abeta peptide can be useful for a diagnostic method based on the detection of soluble abeta peptides in CSF or blood. The authors of the present invention have developed anti-Abeta peptide specific antibodies which allow the detection of the Abeta peptide both in peripheral tissues as well in amyloid plaques present in the central nervous system. These antibodies can then be used for the diagnosis of these types of diseases. In view of the high degree of conservation in the amyloid peptide sequences among different species, the antibodies are useful not only for the diagnosis of amyloid diseases in dogs but also in other non-human mammals as well as in dogs.

Thus, in another aspect, the invention relates to a method for the diagnosis of a disease associated with amyloid deposition in a subject comprising contacting a biological sample from said subject with an antibody or antibody mixture wherein said antibody or antibody-mixture comprises an antibody raised against a peptide having the sequence of SEQ ID NO:3 and detecting the formation of immune complexes between said antibody and at least an amyloid-containing component within the sample.

The term “diagnosis”, as used herein, generally includes determination of a subject's susceptibility to a disease or disorder, determination as to whether a subject is presently affected by a disease or disorder, prognosis of a subject affected by a disease or disorder.

The expression “disease associated with amyloid deposition” has been defined in detailed above and relates to a series of alterations occurring both in human and non-human mammals resulting from the formation of amyloid deposits in different parts of the body. The diseases are selected from the group of localised amyloidosis, systemic amyloidosis, secondary amyloidosis, scrapie, bovine spongiform encephalitis, Creutzfeld-Jacob disease, Alzheimer's disease, cerebral amyloid angiopathy, involutive depression, confusional syndrome, dysthymia, cognitive dysfunction syndrome (CDS) and amyloid-related tissue degeneration.

The term “subject”, as used herein, relates to refers to any individual or patient to which the subject methods are performed and includes, without limitation, humans and non-humans such as dogs, cats, monkeys, bears, rabbits, cows, sheep, pigs, guinea pigs, dolphins and Iberian lynxes.

The term “biological sample”, as used herein, relates to a sample of tissue or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells, organs, biopsies and also samples of in vitro cell culture constituents including but not limited to conditioned media resulting from the growth of cells and tissues in culture medium. In a preferred embodiment, the biological sample is selected from the group of a biofluid or a sample from the central nervous system. In a still more preferred embodiment, the biofluid is selected from the group of blood, serum and CSF. In another preferred embodiment, the biological sample is a sample from the central nervous system. In a more preferred embodiment, the sample from the central nervous system is a brain biopsy.

In the case that the diagnostic method is to be carried out on a biofluid, then the amyloid peptide in said biofluid can be carried out using any known immunoassay. For example, when the CSF, plasma, serum, brain tissue homogenate or brain tissue extract is used as a sample, the immunoassay may be conducted by a solid phase enzyme-linked immunosorbent assay (hereinafter often referred to as “solid phase ELISA”) or an immunoblotting method. Examples of immunoblotting methods include dot blotting method, Western blotting method and the like. With respect to the brain tissue section, the immunoassay may be conducted by a customary immuno-histochemical method.

The solid phase ELISA may be conducted as follows. A sample such as CSF, plasma, serum and brain tissue extract is put in a well of, for example, a polystyrene-made microplate and, then, a buffer such as a carbonate-bicarbonate buffer or a phosphate buffer is put in the well. The mixture is stationarily incubated for a predetermined period of time so that proteins in the sample are adsorbed on the inner surface of the well. The incubation is generally conducted in a carbonate-bicarbonate buffer at 4 DEG C overnight. The incubation may also be conducted at room temperature for 2 hours, or at 37 DEG C for 1 hour. Then, the plate is washed with a phosphate buffer. The washing may also be conducted using a buffer containing a surfactant. After the washing of the plate, the antibody of the present invention is put in the well as a primary antibody, and the plate is incubated to advance an antigen-antibody reaction. Then, the plate is washed with a buffer, and a solution containing a predetermined concentration of an enzyme-labeled anti-immunoglobulin (Ig) antibody is added in the well as a secondary antibody to react with the antibody of the present invention which has been reacted with an antigen adsorbed on the plate. Then, the plate is washed, and a substrate such as o-phenylenediamine is added to the plate to determine the enzyme activity of the reaction mixture. It is also possible that instead of the enzyme-labeled anti-Ig antibody, a biotin-labeled antibody is used for reacting with the antibody of the present invention and, then, an enzyme-labeled avidin is reacted with the biotin-labeled antibody (ABC method). Further, the solid ELISA may also be conducted by a customary peroxidase-antiperoxidase (PAP) method. The enzyme activity determined indirectly represents the amount of the portion of a human brain senile plaque amyloid precursor protein, having a protease inhibiting activity present in the sample employed.

In practicing the solid phase ELISA, a fluorescent substance-labeled or a radioisotope-labeled anti-mouse Ig antibody may also be used instead of the above-mentioned enzyme-labeled anti-Ig antibody.

Further, the solid phase ELISA may also be conducted by the so-called sandwich immunoassay method in which two types of antibodies which are capable of recognizing different epitopes, are used. Particularly, one monoclonal antibody is adsorbed on a microplate as a primary antibody. Then, a sample is applied to the plate to react an antigen in the sample with the primary monoclonal antibody adsorbed on the plate. Then, the other monoclonal antibody of the present invention is reacted with the antigen which has been reacted with the primary antibody. Then, a secondary antibody as mentioned above is reacted with the antigen-monoclonal antibody complex. This method is advantageous from the standpoint of sensitivity.

The dot blotting may be conducted as follow. A small amount (generally 0.2 to 10 mu 1) of a sample such as CSF, plasma, serum, brain tissue extract and brain tissue homogenate is dotted on a nitrocellulose filter or a nylon filter. The filter is subjected to blocking with a bovine serum albumin (hereinafter referred to as “BSA”), a gelatin, a skim milk or the like in a buffer such as a Tris-HCl buffer. Then, the antibody of the present invention is reacted with the antigen on the filter. After completion of the reaction, the resultant filter is washed with a buffer. Then, an enzyme-labeled anti-Ig antibody is applied to the filter as a secondary antibody to react the secondary antibody with the antibody of the present invention. The filter is washed. Then, to the filter is added a substrate, and the enzyme activity is determined based on the thickness of color assumed by the enzyme-substrate reaction. Examples of substrates include 4-chloronaphthol, diaminobenzidine and the like. As the secondary antibody, a labeled protein A may also be used instead of the above-mentioned anti-Ig antibody.

The Western blotting may be conducted as follow. A sample such as CSF, plasma, serum, brain tissue extract, brain tissue homogenate as such or after being boiled with sodium dodecyl sulfate (hereinafter referred to as “SDS”), is subjected to polyacrylamide gel electrophoresis (hereinafter referred to as “PAGE”). The resultant gel is equilibrated with a transfer buffer and, then, the protein in the gel is electrotransferred into a nitrocellulose filter or a nylon filter. The thus obtained filter is treated in the same manner as in the case of the above-mentioned dot blotting to thereby determine the amount of the protein comprising at least a portion of a human brain SPAP in the sample.

In practicing the above-mentioned immunoassay, the antibody of the present invention may be labeled with a dye such as rhodamine and fluorescein isothiocyanate (FITC); an enzyme such as peroxidase; or a radioactive isotope, so that the assay can be conducted without using a secondary antibody.

The antiserum or the fraction of the polyclonal antibody obtained by the above-mentioned purification may generally be diluted to an appropriate degree and the dilution is used for immunoassay. In the case of the solid phase ELISA, the antiserum may generally be diluted about 100 to about 4000 times, preferably about 200 to about 1000 times. In the case of the Western blotting, the antiserum may generally be diluted about 100 to about 2000 times, preferably about 200 to about 1000 times. In the case of the solid phase ELISA, the antiserum or the fraction containing the polyclonal

The immuno-histochemical assay of a brain tissue section sample may be conducted by a customary method as follow. The brain of a dead patient is frozen, or fixed with formalin and embedded in paraffin. From the thus obtained brain, a tissue section is prepared. The resultant tissue section is subjected to assay using the antibody of the present invention by the above-mentioned ABC method or a peroxidase-antiperoxidase (PAP) method. In the PAP method, diaminobenzidine is generally used as a substrate of peroxidase.

In the case that the diagnosis is to be carried out in a sample from the central nervous system, the detection is preferably carried out by immunohistochemical analysis. For the purposes of immunohistochemical analysis, the biopsied sample may be treated with trypsin (for instance, with 0.1% aqueous trypsin solution at 37 DEG C for 10 min). However, the trypsin treatment is not always required. The sample may then be fixated for brief periods of time. The purpose of such brief fixation is to avoid conformational alterations from prolonged fixation that could destroy the antigenic sites on the beta-AP-containing molecules in the samples. For example, the samples can be fixed for 15-60 minutes in 10% neutral buffered formalin, following which the sample is removed to a physiological buffer (e.g., phosphate buffered normal saline, pH 7.6, with 0.02% sodium azide) and stored at 4 degrees C. Alternatively, a longer period of fixation can be used, but in the preferred embodiments of this invention, brief fixation (i.e., fixation for generally less than three hours) is called for. Other fixatives can be used including a picric acid-containing fixative (e.g., Bouin's method) or the PLP fixative (periodate-lysine-paraformaldehyde).

After this treatment, the fixed sample can be further prepared for immunohistologic staining. For example, the sample can be embedded in a medium such as paraffin and sectioned in a microtome, and typically, 5-15 mu m-thick sections can be mounted on glass microscope slides for immunocytochemistry. Alternatively, a microtome or a cryostat can be used to prepare frozen sections from either a fresh-frozen or a fixed-frozen sample of skin, intestine, or other non-neural tissue. The inclusion of some subcutaneous tissue in the skin sample is often useful diagnostically, since perivascular amyloid deposits can be seen to advantage in this region.

Detection of the amyloid deposits in the formalin-fixed tissue involves incubating formalin-fixed tissue with a solution containing the diagnostic antibodies of the invention. Upon incubation, the antibody binds to the amyloid peptide present in the amyloid deposits in the tissue. The antibody-antigen complex may be visualized using a second antibody directed to the antibody of the invention and being labeled according to methods known in the art (see, e.g. Harlow and Lane, Antibodies, A Laboratory Manual, Cold Spring Harbor Laboratory, Cold spring Harbor, 1988.)

In another aspect, the invention provides methods of in vivo imaging amyloid deposits in a patient. Such methods are useful to diagnose or confirm diagnosis of Alzheimer's disease, or susceptibility thereto. For example, the methods can be used on a patient presenting symptoms of dementia. If the patient has abnormal amyloid deposits, then the patient is likely suffering from Alzheimer's disease. The methods can also be used on asymptomatic patients. Presence of abnormal deposits of amyloid indicates susceptibility to future symptomatic disease. The methods are also useful for monitoring disease progression and/or response to treatment in patients who have been previously diagnosed with Alzheimer's disease.

The methods work by administering a reagent, such as antibody that binds to Abeta, to the patient and then detecting the agent after it has bound. Such antibodies typically bind and may or may not induce a substantial clearing response. The clearing response can be avoided by using antibody fragments lacking a full-length constant region, such as Fabs. In some methods, the same antibody can serve as both a treatment and diagnostic reagent. Diagnostic reagents can be administered by intravenous injection into the body of the patient, or directly into the brain by intracranial injection or by drilling a hole through the skull. The dosage of reagent should be within the same ranges as for treatment methods. Typically, the reagent is labeled, although in some methods, the primary reagent with affinity for Amyloid beta is unlabelled and a secondary labelling agent is used to bind to the primary reagent. The choice of label depends on the means of detection. For example, a fluorescent label is suitable for optical detection. Use of paramagnetic labels is suitable for tomographic detection without surgical intervention. Radioactive labels can also be detected using PET or SPECT.

Diagnosis is performed by comparing the number, size, and/or intensity of labelled loci, to corresponding baseline values. The base line values can represent the mean levels in a population of undiseased individuals. Baseline values can also represent previous levels determined in the same patient. For example, baseline values can be determined in a patient before beginning treatment, and measured values thereafter compared with the baseline values. A decrease in values relative to baseline signals a positive response to treatment.

The invention will be described by way of the following examples which are to be considered as merely illustrative and not limitative of the scope of the invention.

### EXAMPLES

### Example 1

### Synthesis of Peptide Sequences of the Present Invention

Two of the peptide sequences of the present invention, SEQ ID NO 1 and SEQ ID NO 3, corresponding to the canine Abeta protein from amino acids 1 to 40 (Abeta 1-40) and amino acids 28 to 40 (Abeta 28-40), respectively, were synthesized individually by the Merryfield solid-phase synthesis technique on Applied Biosystems automated peptide synthesizer (Model 433A Peptide Synthesizer) using Fluorenylmethyloxycarbonyl chloride (Fmoc) chemistry as protecting groups. After complete assembly of the desired peptide, the product was treated with trifluoroacetic acid to cleave the peptide from the resin where it was attached and deblock the protecting groups on the amino acid side chains. The cleaved, extracted and washed peptides were purified by HPLC using a Waters Delta Prep 4000 HPLC analyzer, and characterized by reverse phase HPLC with a Waters Delta 6000 HPLC apparatus. Finally the total amount of peptides was calculated by Mass Spectrometry by using an Applied Biosystems apparatus, model 4700 Proteomic analyzer.

In order to enhance its immunogeneicity, each peptide was additionally conjugated with Keyhole Limpet Hemocyanin (KLH), a standard carrier widely used in vaccine administration procedures. The resultant amount of KLH-conjugated peptide was split into aliquots of 0.5 mg peptide and 0.7 mg KLH each, lyophilized and stored at −20° C.

### Example 2

### Evaluation of the Immunogenicity of the Peptide Sequences of the Present Invention

To design a veterinary vaccine composition that generates a high level of high affinity antibodies with cross reactivity to the soluble Abeta 1-40 peptide and the plaques in the CNS of dogs with CDS, the relative immunogeneicity of the synthesized sequences of example 1 were characterized separately. To do that, SEQ ID NO 1 and SEQ ID NO 3 were administered conjugated with KLH and mixed with 1 mg of Alum (Sigma, St Louis, U.S.A.) an adjuvant of general veterinary use. The relative immunogenicity of the two peptide sequences was compared according to standard titration of the immune products generated.

Experiments were performed in adult female New Zealand White rabbits weighing between 1.5-2 kg at the beginning of the study. They were kept on a 12 h light/12 h dark cycle and housed with free access to food and water. All animals were manipulated according to the European legislation (86/609/EEC) for animal handling and experimentation. Procedures were approved by the Ethic Committee of the Universitat de Barcelona, under supervision of the Generalitat de Catalunya; all efforts were made to minimize animal suffering and to use only the number of animals necessary to produce reliable scientific data.

Rabbits were subcutaneously immunized with 100 μg of peptide from either: a) SEQ ID NO 3 conjugated with KLH and 1 mg Alum used as adjuvant (n=2); b) SEQ ID NO 1 conjugated with KLH and 1 mg Alum used as adjuvant (n=2); or c) KLH and 1 mg Alum (no peptide n=2). The immunization protocol consisted of a dose subcutaneously delivered up to six sites near the back of the neck, and repeated at days 11, 21, 31 and 41.

Samples of serum were collected from each rabbit at days 0, 21, 31 and 51. Each time, 5 ml blood samples were collected from the ear marginal vein in plastic tubes with no additive. At day 71 rabbits were anaesthetized and exanguinated. Blood samples were then kept at room temperature (RT) for 30 min, afterwards they were centrifuged for 10 min at 3000 rpm. Supernatants were collected as serum samples, aliquoted and stored at −80° C.

Titration of serum samples to detect the presence of antibodies that had been raised by SEQ ID NO 1 and SEQ ID NO 3 was performed by Enzime-Linked Immunosorbent Assays (ELISA) on 96-well plates developed by the biotin-avidin-peroxidase method. Plate sensitization was made overnight at 4° C. with 2 μg/ml of Abeta 1-40 peptide diluted in 50 mM NaHCO₃, pH 9.6. After washing, plate blockade was performed with 0.05% PBS-T and 5% powdered milk for 2 h at RT. Serum samples were applied at 4 different dilutions (1/100, 1/1000, 1/10000 and 1/100000) in PBS for 2 hours at RT. After washing, specific binding was detected by incubation with Biotin conjugated anti-Rabbit IgG antibody (Sigma, St. Louis, Mo., U.S.A) diluted 1/20000 in TBS for 1 hour at RT. After washing and incubation with ExtrAvidin (Sigma, St. Louis, Mo., U.S.A) samples were developed with tetramethylbenzidine for 10 min in the dark. Plates were densitometrically analyzed at 450 nm with a Tecan Sunrise Absorbance Reader apparatus (Tecan Iberica, Madrid, Spain). Serum samples from day 0 (pre-immunized rabbit samples) were used as negative controls. Monoconal rabbit anti-Abeta 1-40/1-42 antibodies (Chemicon, Temecula, Calif., USA) diluted 1/10000 in TBS were used as positive controls. Standarization of ELISA detections was performed by construction of a patron curve using Rabbit IgG at 4 dilutions (1/5, 1/25, 1/125 and 1/625). To assess the plate sticking, no sensitization of 12 wells was made in each plate. In all cases, serum antibody titration was performed by duplicated.

No antibodies were detected in any serum samples of control rabbits (treated with KLH and Alum) at any time-point. In these animals, the highest values of optic density were found at day 31 but they were similar to background.

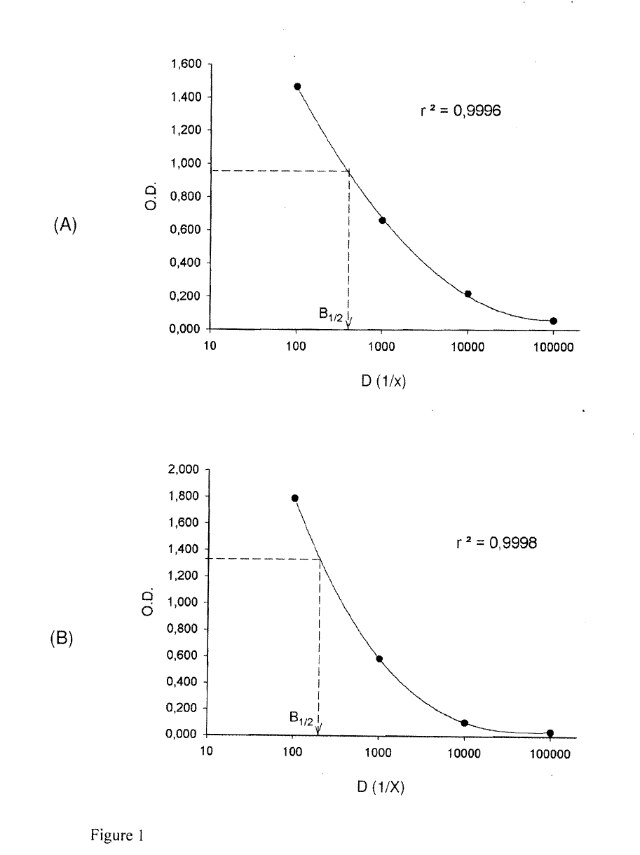

As can be deduced from FIG. 1, from day 21 following the first immunization, a specific production of antibodies was detected in serum samples of rabbits injected with SEQ ID NO 3 (FIG. 1A). The highest levels of immunization were detected in samples of day 31. Similar results were found in serum samples of rabbits immunized with the SEQ ID NO 1 (FIG. 1B). Titration of antibodies from day 31 indicates that, for the SEQ ID NO 3, the serum dilution that presents half of the maximal binding (B_{1/2}) is the 1/611 one, whereas this value for SEQ ID NO 1 is detected at a dilution of 1/240. This demonstrates that, in rabbits, the SEQ ID NO 3 presents a higher immunogenic response than the SEQ ID NO 1. (FIG. 1).

### Example 3

### Purification of High Affinity Antibodies from Rabbit Serum

Together with the high level of high affinity antibodies cross-reacting with the canine Abeta 1-40 (SEQ ID NO 1) and Abeta 28-40 (SEQ ID NO 3), serum samples generally also present antibodies against immunogens and proteins not specifically related to an Abeta immune response. Thus, to assess the potential therapeutic efficacy in the clearance of soluble Abeta peptide and amyloid plaques in the dog CNS, it is firstly needed to specifically isolate the high affinity anti-Abeta 28-40 (or anti-SEQ ID NO 3) and anti-Abeta1-40 (or anti-SEQ ID NO 1) antibodies from these samples.

Antibody purification was made by affinity chromatography in columns of 4B Sepharose activated with BrCN (GE healthcare 15gr) according to standard procedures. Synthetic Abeta1-40 peptide (SEQ ID NO 1) was linked to the BrCN and used as antigen for the affinity purification of antibodies. Anti-Abeta28-40 antibodies were purified from rabbit serum samples presenting the highest level of titers, i.e. serum samples collected on day 31 after immunization (example 2). The same was true for serum samples used for purification of anti-Abeta 1-40 antibodies. Purified antibodies were firstly eluted with an acid solution of 100 mM glycine in PBS (pH 2.5) and, then with a basic solution of 100 mM glycine and 4 M urea in PBS (pH 9)

The purified antibodies were quantified by ELISA on 96-well plates developed by the biotin-avidin-peroxidase method. Plate sensitization was made overnight at 4° C. with 2n/ml of canine Abeta 1-40 peptide diluted in 50 mM NaHCO₃ pH 9.6. The ELISA procedure was then carried out as explained in example 2.

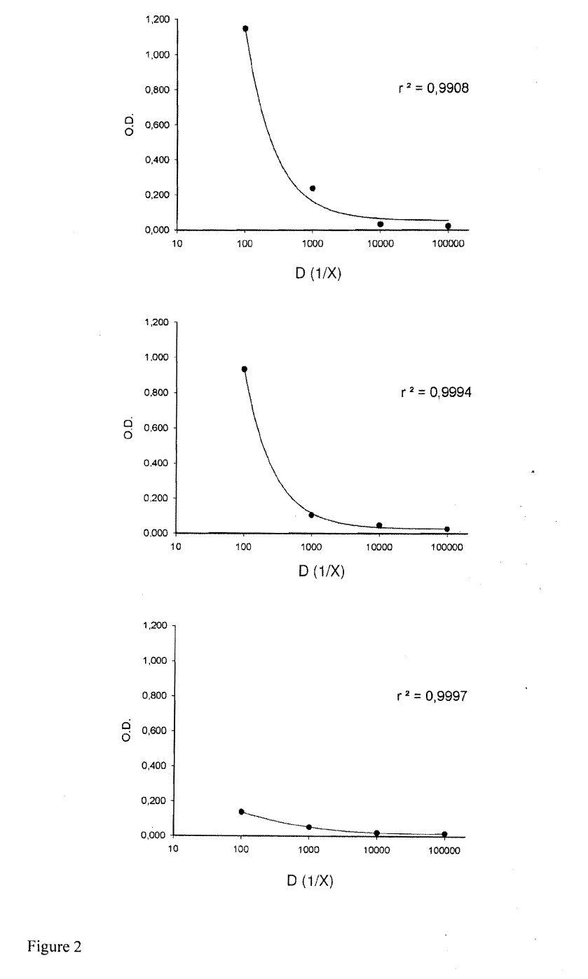

Titration of purified sera from rabbits immunized with canine Abeta 28-40 (SEQ ID NO 3) or with canine Abeta 1-40 (SEQ ID NO 1) peptides was compared with purification of serum obtained from rabbits only immunized with the adjuvant and the carrier (FIG. 2 A-C). High titers of specific antibodies against canine Abeta1-40 were detected in serum from rabbits immunized with the Abeta 28-40 fragment (SEQ ID NO 3), as can be deduced from FIG. 2 (A), which corresponds to the titration of purified sera from rabbits immunized with this peptide sequence. Although slightly lower than the acid elution, high titers of anti-Abeta antibodies were also detected in the basic elution of the purification of Abeta 28-40 serum. Similar results were found in the purification of serum samples from rabbits immunized with the Abeta 1-40 peptide (SEQ ID NO 1), as derived from FIG. 2 (B), which is the titration of purified sera from rabbits immunized with peptide sequence SEQ ID NO 1.

Taken together, results from example 2 and example 3 clearly demonstrates that a standard pharmacological preparation of Abeta 28-40 peptide sequence (SEQ ID NO 3) or Abeta 1-40 peptide sequence (SEQ IN DO 1), formulated as a vaccine, induces in each case the production of a highly specific antibody against the canine Abeta 1-40 peptide.

### Example 4

### Evaluation of Purified Anti-Abeta Peptide Antibodies by Cross-Reactivitiy to Canine Soluble Abeta

To validate the two generated veterinary vaccine compositions of Example 3, which generate a high level of high affinity anti-Abeta antibodies with cross reactivity to the soluble Abeta peptide, it is necessary to evaluate the antigen cross-reactivity of the purified antibodies with the canine soluble Abeta. Thus, the purified anti-Abeta 28-40 and anti-Abeta 1-40 antibodies were separately used for immunodetection of canine soluble Abeta 1-40 peptide by dot blot experiments as follow.

Canine soluble Abeta peptide at a concentration of 2 μg/cm² was directly incubated onto 0.2 μg pore polyvinylidene difluoride (PVDF) membranes (Amersham, UK). Membranes were washed in 10 mM Tris-buffered saline with 0.1% Tween 20 (TTBS). After the 1 h blocking step performed in the same buffer containing 5% milk/TTBS, and allowed to air dry for 20 min. Then 2 μl dots of purified anti-Abeta 1-40 peptide and anti-Abeta 28-40 peptide antibodies were separately blotted onto the activated membrane for 30 min. After washing with the previous buffer, an horseradish peroxidase-conjugated IgG goat anti-rabbit was applied at 1:10,000 (Bio-Rad, Hercules, Calif.) as secondary antibody and densitometricaly developed with the ECL Amersham kit.

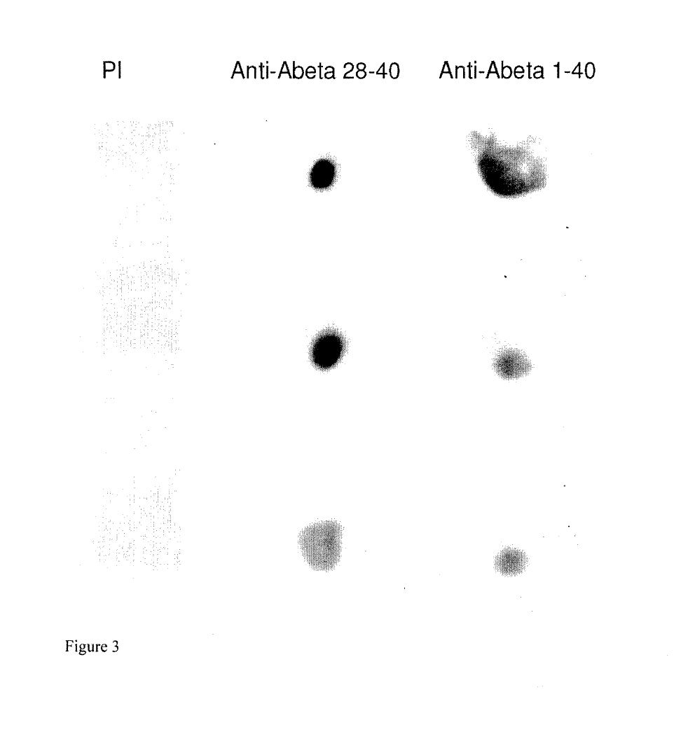

As shown in FIG. 3 canine soluble Abeta 1-40 peptide was detected with both the purified Anti-Abeta 28-40 peptide (or anti-SEQ ID NO 3) and Anti-Abeta 1-40 peptide (or anti-SEQ ID NO 1) antibodies, whereas membrane dots incubated with the rabbit pre-immune serum showed no specific Abeta immunolabelling.

Thus, the antibodies raised in this invention can be included in a pharmaceutical formulation suitable for administration to a canine subject, which provides an effective method for immunoneutralization of soluble Abeta proteins in the CNS for the prevention and treatment of CDS in the dog and other non-human mammals that have Abeta proteins with high homology to that of dogs, such as cats, monkeys, polar bears, rabbits, cows, sheep, pigs, guinea pigs, dolphins and Iberian lynxes. As explained previously, the induction of plasma anti-Abeta or other antipeptide antibodies serves to quickly and efficiently increase the clearance of CNS Abeta. Generation of this peripheral sink mechanism is considered useful for treating abnormal Abeta protein accumulation in the CNS, because of the resulting net efflux of central Abeta to the periphery and its rapid elimination.

### Example 5

### Evaluation of Purified Antibodies by Cross-Reactivities to Diffuse Amyloid Plaques of Aged Canine with CDS

Male and female dogs of various breeds and ages certified by their medical records from the veterinary hospital Ars Veterinaria of Barcelona, Spain, were used in this study. In all cases, donation was formally approved by the owner and euthanasia justified for medical reasons; the animals were sacrificed with an I.V. overdose of sodium thiopental (75 mg/kg; Thiobarbital; Braun Medical S.A.). All animals were treated according to European legislation on animal handling and experiments (86/609/EU), and procedures were approved by the Ethical Committee of the University of Barcelona.

All efforts were made to minimize animal suffering and to use no more than the number of animals needed for reliable scientific data.

To evaluate the cognitive status of the dogs, a validated cognitive test was used in collaboration with pet owners. This test includes nine items: elimination behavior, life rhythm, walking, posture, playful and exploratory behaviors, interaction with other animals or with the owners, learning of specific behaviors and adaptive capabilities and was filled out by a veterinarian. For each item score 1 indicated the normality of the specific behavior, and scores 2, 3, 4 and 5 the degree of abnormal behavior. The final total score reflected the cognitive status of the animal. In some cases normality was qualified considering the diagnostic previously established by the veterinarian. Based on their score, dogs were categorized as young control, i.e. with no signs of cognitive disorder, light cognitive impairment and severe cognitive impairment. These signs are typical disorders for CDS.

Immediately after death, dog brains were quickly removed, and 1-cm-thick coronal sections of cerebral cortex were immersion fixed in 10% neutral buffered formalin for 4 weeks. After a 3-day cryoprotection, they were frozen on powdered dry ice, and 12-μm-thick serial sections of dorsal anterior prefrontal cortex corresponding to area 8a on the proreal gyrus were obtained with a cryostat. Once mounted on i-coated glass slides, they were kept at −40° C. until use.

Series of sections of each brain were processed for immunohistochemical studies with the avidin-biotin complex (ABC) method employing the Vectastain Elite ABC kit (Vector Laboratories, Burlingame, Calif.). To unmask plaque immunoreactivity, sections were pretreated with 98-100% formic acid for 3 min at RT. Samples of purified anti Abeta antibodies from example 3 were used at dilutions of 1/100. For negative controls, sections were stained with omission of this antibody. After rinse, sections were incubated in 0.3% hydrogen peroxide in methanol for 10 min at RT to block endogenous peroxidases. Afterward, they were incubated in an immunobuffer solution of 3% normal goat serum (NGS) with 0.5% Triton X-100 in PBS for 1 hr at RT to block nonspecific reactions. Then, sections were incubated with the purified antibody overnight at 4° C. Biotilynated goat anti-rabbit-IgG (1/200; Vector Laboratories) was used. Visualization of the antigen-antibody complex was performed with 1/100 dilution of avidin-biotin peroxidase with 3,3 α-diaminobenzidine peroxidase substrate kit (Vector Laboratories, U.S.A.). Adjacent brain sections were stained with the Bielschowsky's silver staining in order to reveal the presence of diffuse amyloid plaques and validate the specificity of the purified antibodies. Also, additional adjacent sections were counterstained with cresyl violet to reveal the canine brain cytoarchitecture.

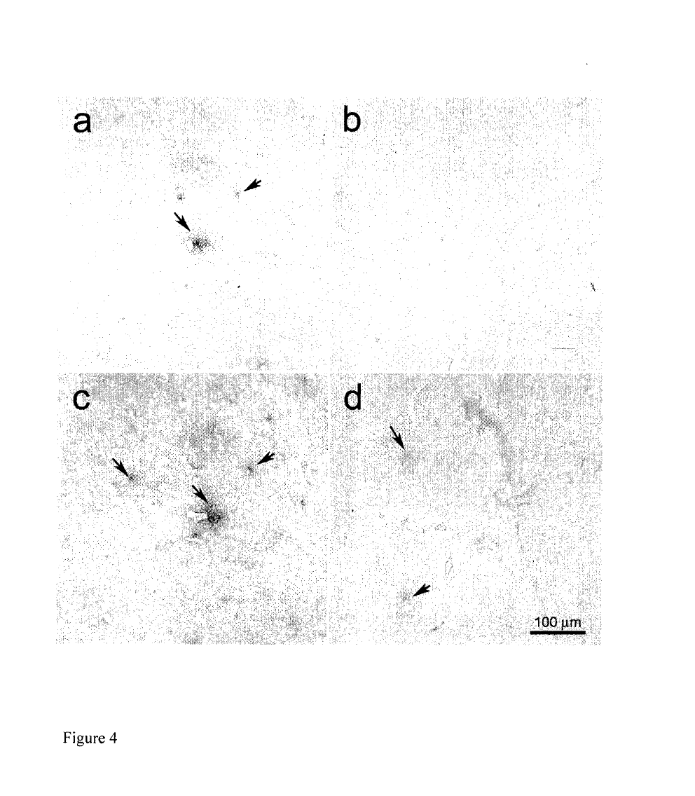

Purified anti-Abeta antibodies raised against Abeta 28-40 and from Abeta 1-40 fragments specifically stained Abeta diffuse plaques in the dog brain sections, as can be seen in images c) and a), respectively, of FIG. 4. Brain parenchyma and neurons showed no specific staining. Control staining with the Bielschovsky's method revealed a very similar staining to the one observed by immunohistochemistry. The observed differences are explained by the known limitations of histological stainings (such as the Bielschowsky's one) of immature Abeta plaques of the canine brain (Shimada et al 1991). These results constitute clear pieces of evidence for the specificity of the raised and purified antibodies of the invention.

From FIG. 4 b), which corresponds to the immunohistochemistry with pre-immune serum of the dog brains, it is deduced that the serum was not able to detect any Abeta plaque. Low immunoreactivity against canine plaques (arrows) was detected with commercial anti-human Abeta peptide antibody, as shown in FIG. 4 d).

Starting at the age of 8 years, and increasing with age and cognitive impairment severity, Abeta immunohistochemistry revealed the specific anti-Abeta 28-40 and anti-Abeta 1-40 antibodies binding to delicate and progressively more compact diffuse deposits. The deposits density and distribution throughout all cortical layers of the cerebral cortex coincide with the well-known four-stage distribution (I-IV) of the canine maturation process of diffuse amyloid plaques (Pugliese et al., 2004, J. Neurosci. Res. 77:913-920). No plaques were detected in subcortical brain areas or the white matter.

### Example 6

### Synthesis of High Affinity Monoclonal Anti-Abeta Peptide Antibodies

An alternative therapy to active immunization with Abeta peptide for the treatment and/or prevention of cognitive deficits in non-human mammals is the passive administration of specific anti-Abeta antibodies. Passive immunotherapy allows customizing the antibody molecule to obtain one aimed at a specific epitope, and select the isotype of the antibody to be administered. Another advantage of the direct administration of a specific antibody is that its effectiveness does not depend on the immune system activation that is generally very low in aged animals. Another advantage of the no involvement of the immune system is that, in case of adverse reactions, passive immunotherapy immediate suspension or reduction of the treatment will directly stop these adverse effects.

Monoclonal anti-Abeta antibodies raised by the SEQ ID NO 3 of the present invention, corresponding to the canine Abeta protein from amino acids 28 to 40 (Abeta 28-40), were synthesized by fusing myeloma cells with the spleen cells from a mouse that has been immunized with SEQ ID NO 3.

Experiments were performed in adult male BALB/c mice weighing between 20-30 g at the beginning of the study. They were kept on a 12 h light/12 h dark cycle and housed with free access to food and water. All animals were manipulated according to the European legislation (86/609/EEC) for animal handling and experimentation. Procedures were approved by the Ethic Committee of the Universitat de Barcelona, under supervision of the Generalitat de Catalunya; all efforts were made to minimize animal suffering.

3 mice were immunized intraperitoneally with 0.25 mg/mL SEQ ID NO 3 in the presence of Freund's complete adjuvant (BD, U.S.A.). Subsequent boosters with SEQ ID NO 3 (0.25 mg/mL) mixed with Freund's incomplete adjuvant (BD, U.S.A.) were carried out in the interval of two weeks until a strong immune response to the antigen was recognized. Sera from mice were periodically collected in order to determine the immune response titer in each serum as explained in example 2.

For hybridoma construction, the lymphocytes (1×10⁸ cells) collected from the spleen of the immunized mouse showing the best antibody titer were fused with the Sp2/0-Ag14 myeloma cell line using PEG fusion method. The hybrid cells were transferred into a 96-well plate containing HAT medium with the feeder layers of mouse peritoneal macrophage. For screening of positive hybridoma cells, the culture broth was subjected to modified competitive direct ELISA as explained in example 3. The hybridomas with relatively high titers were cloned by limiting dilution and then enriched.

Antibody purification was made by affinity chromatography in columns of 4B Sepharose activated with BrCN (GE healthcare 15gr) according to standard procedures explained in example 3.

The purified antibodies were quantified by ELISA on 96-well plates developed by the biotin-avidin-peroxidase method. Plate sensitization was made overnight at 4° C. with 2 μg/ml of canine Abeta 1-40 peptide diluted in 50 mM NaHCO₃ pH 9.6. The ELISA procedure was then carried out as explained in example 2.

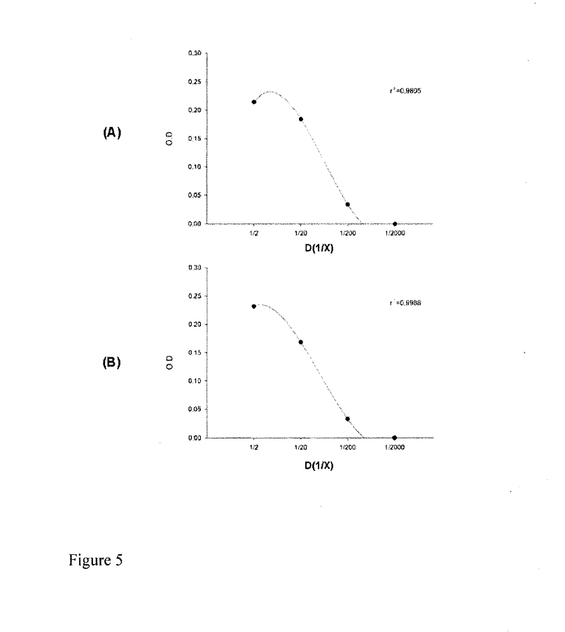

Immunoreactivity of purified monoclonal anti-Abeta antibody was assessed by antibody titration with canine Abeta 28-40 (SEQ ID NO 3) or with canine Abeta1-40 (SEQ ID NO: 1 (FIG. 5 A-B). High titers of specific antibodies against canine Abeta1-40 were detected in purified monoclonal antibodies with the Abeta 28-40 fragment (SEQ ID NO 3), as can be deduced from FIG. 5 (A), which corresponds to the titration of purified monoclonal antibodies with this peptide sequence. Similar results were found when titration was performed against the Abeta 1-40 peptide (SEQ ID NO 1), as derived from FIG. 5 (B), which is the titration of the purified monoclonal antibody with peptide sequence SEQ ID NO 1.2

Taken together, these results clearly demonstrate that a standard pharmacological preparation of mouse monoclonal antibody raised against the Abeta 28-40 peptide sequence (SEQ ID NO 3), presents a highly specific reactivity against the canine Abeta 1-40 peptide.

### Example 7

### Evaluation of Monoclonal Anti-Abeta Antibodies by Cross-Reactivitiy to Canine Soluble Abeta

To validate the generated veterinary composition of Example 6, which contains high level of high affinity anti-Abeta antibodies with cross reactivity to the soluble Abeta peptide, it is necessary to evaluate the antigen cross-reactivity of the purified antibodies with the canine soluble Abeta. Thus, the purified monoclonal anti-Abeta 28-40 antibody was separately used for immunodetection of canine soluble Abeta 28-40 and Abeta 1-40 peptides by dot blot experiments as follows.

Canine soluble Abeta 28-40 and Abeta1-40 peptides at a concentration of 2 ng/cm² each were separately incubated onto 0.2 μg pore polyvinylidene difluoride (PVDF) membranes (Amersham, UK). Membranes were washed in 10 mM Tris-buffered saline with 0.1% Tween 20 (TTBS). After the 1 h blocking step performed in the same buffer containing 5% milk/TTBS, and allowed to air dry for 20 min. Then 2 μl dots of purified monoclonal anti-Abeta 28-40 antibody were blotted onto each activated membrane for 30 min. After washing with the previous buffer, a horseradish peroxidase-conjugated IgG rabbit anti-mouse was applied at 1:10,000 (Bio-Rad, Hercules, Calif.) as secondary antibody and densitometricaly developed with the ECL Amersham kit.

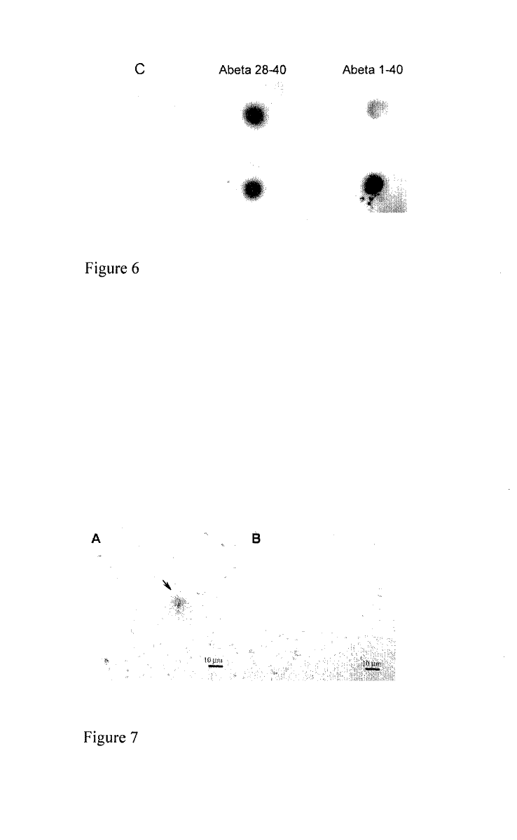

As shown in FIG. 6 purified monoclonal anti Abeta 28-40 antibody (or anti-SEQ ID NO 3) detected both canine soluble Abeta 28-40 peptide (Abeta 28-40 in FIG. 6) and Abeta 1-40 peptide (Abeta 1-40 in FIG. 6) whereas membrane dots incubated with bovine serum albumin (C in FIG. 6) showed no specific immunolabelling.

Thus, the antibodies synthesized in this invention can be included in a pharmaceutical formulation suitable for administration to a canine subject, which provides an effective method for immunoneutralization of soluble Abeta proteins in the CNS for the prevention and treatment of CDS in the dog and other non-human mammals that have Abeta proteins with high homology to that of dogs, such as cats, monkeys, polar bears, rabbits, cows, sheep, pigs, guinea pigs, dolphins and Iberian lynxes. As explained previously, the induction of plasma anti-Abeta or other antipeptide antibodies serves to quickly and efficiently increase the clearance of CNS Abeta. Generation of this peripheral sink mechanism is considered useful for treating abnormal Abeta protein accumulation in the CNS, because of the resulting net efflux of central Abeta to the periphery and its rapid elimination.

### Example 8

### Evaluation of Monoclonal Antibodies by Cross-Reactivities to Diffuse Amyloid Plaques of Aged Canine with CDS

Male and female dogs of various breeds and ages certified by their medical records from the veterinary hospital Ars Veterinaria of Barcelona, Spain, were used in this study. In all cases, donation was formally approved by the owner and euthanasia justified for medical reasons; the animals were sacrificed with an I.V. overdose of sodium thiopental (75 mg/kg; Thiobarbital; Braun Medical S.A.). All animals were treated according to European legislation on animal handling and experiments (86/609/EU), and procedures were approved by the Ethical Committee of the University of Barcelona. All efforts were made to minimize animal suffering and to use no more than the number of animals needed for reliable scientific data.

To evaluate the cognitive status of the dogs, a validated cognitive test was used in collaboration with pet owners. This test includes nine items: elimination behavior, life rhythm, walking, posture, playful and exploratory behaviors, interaction with other animals or with the owners, learning of specific behaviors and adaptive capabilities and was filled out by a veterinarian. For each item score 1 indicated the normality of the specific behavior, and scores 2, 3, 4 and 5 the degree of abnormal behavior. The final total score reflected the cognitive status of the animal. In some cases normality was qualified considering the diagnostic previously established by the veterinarian. Based on their score, dogs were categorized as young control, i.e. with no signs of cognitive disorder, light cognitive impairment and severe cognitive impairment. These signs are typical disorders for CDS.

Immediately after death, dog brains were quickly removed, and 1-cm-thick coronal sections of cerebral cortex were immersion fixed in 10% neutral buffered formalin for 4 weeks. After a 3-day cryoprotection, they were frozen on powdered dry ice, and 12-μm-thick serial sections of dorsal anterior prefrontal cortex corresponding to area 8a on the proreal gyrus were obtained with a cryostat. Once mounted on polyglycine-coated glass slides, they were kept at −40° C. until use.

Series of sections of each brain were processed for immunohistochemical studies with the avidin-biotin complex (ABC) method employing the Vectastain Elite ABC kit (Vector Laboratories, Burlingame, Calif.). To unmask plaque immunoreactivity, sections were pretreated with 98-100% formic acid for 3 min at RT. Samples of purified mouse monoclonal anti-Abeta antibodies obtained as shown in example 6 were used at dilutions of 1/10. For negative controls, sections were stained with omission of this antibody. After rinse, sections were incubated in 0.3% hydrogen peroxide in methanol for 10 min at RT to block endogenous peroxidases. Afterwards, they were incubated in an immunobuffer solution of 3% normal goat serum (NGS) with 0.5% Triton X-100 in PBS for 1 hr at RT to block nonspecific reactions. Then, sections were incubated with the purified antibody overnight at 4° C. Biotilynated goat anti-rabbit-IgG (1/200; Vector Laboratories) was used. Visualization of the antigen-antibody complex was performed with 1/100 dilution of avidin-biotin peroxidase with 3,3 α-diaminobenzidine peroxidase substrate kit (Vector Laboratories, U.S.A.). Adjacent brain sections were stained with the Bielschowsky's silver staining in order to reveal the presence of diffuse amyloid plaques and validate the specificity of the purified antibodies. Also, additional adjacent sections were counterstained with cresyl violet to reveal the canine brain cytoarchitecture.

Purified mouse monoclonal anti-Abeta antibodies raised against the Abeta 28-40 peptide fragment specifically stained Abeta diffuse plaques in the dog brain sections, as can be seen in image A) of FIG. 7. Brain parenchyma neurons and gial cells showed no specific staining. Control staining with the Bielschovsky's method revealed a very similar staining to the one observed by immunohistochemistry. The observed differences are explained by the known limitations of histological stainings (such as the Bielschowsky's one) of immature Abeta plaques of the canine brain (Shimada et al 1991). These results constitute clear pieces of evidence for the specificity of the raised and purified monoclonal antibodies of the invention.

From FIG. 7 B), which corresponds to the immunohistochemistry with no specific primary antibody, is deduced that secondary antibodies and reagents used in the procedure were not able to stain any Abeta plaque. This result demonstrates the specificity of the monoclonal antibodies of the invention.

Starting at the age of 8 years, and increasing with age and cognitive impairment severity, Abeta immunohistochemistry revealed the specific monoclonal anti-Abeta 28-40 antibodies binding to delicate and progressively more compact diffuse deposits. The deposits density and distribution throughout all cortical layers of the cerebral cortex coincide with the well-known four-stage distribution (I-IV) of the canine maturation process of diffuse amyloid plaques (Pugliese et al., (2004), J. Neurosci. Res. 77:913-920). No plaques were detected in subcortical brain areas or the white matter.

Thus as indicated in examples 6, 7 and 8, the monoclonal antibodies raised in this invention can be included in a pharmaceutical formulation suitable for administration to a canine subject, and provide an effective method for the clearance of amyloid plaques in the CNS for the prevention and treatment of CDS in the dog and other non-human mammals. As explained previously, the direct administration of anti-Abeta or other antipeptide antibodies serves to quickly and efficiently increase the clearance of CNS Abeta. Generation of this peripheral sink mechanism is considered useful for treating abnormal Abeta protein accumulation in the CNS, because of the resulting net efflux of central Abeta to the periphery and its rapid elimination.

Similarly, the antibodies can also be used for post-mortem diagnostics and in genetic studies to determine the similarities between the Abeta plaque composition and organization of different species.

### Example 9

### The Immunogenicity of Representative Peptide Sequences of the Invention in Dogs as Immunotherapeutic Agents for CDS

To validate the immunogenicity of the peptide according to the present invention, dogs were immunized with the synthesized Abeta 28-40 peptide (SEQ ID NO 3) and antibody generation was assessed in serum samples by ELISA. To do that, the peptide fragment was administered conjugated with KLH and mixed with 1 mg Alum (Sigma, St. Louis, Mo., U.S.A.), an adjuvant of general veterinary use. The immunogenicity of the Abeta28-40 peptide was compared versus pre-immune serum according to standard titration of the immune products generated.

Male and female dogs of various breeds, sex and ages certified by their medical records from the veterinary hospital Ars Veterinaria of Barcelona, Spain, were used in this study. In all cases, pet owners formally informed approved the treatment of their dogs. All animals were treated according to European legislation on animal experimentation (86/609/EU), and procedures were approved by the Ethical Committee of the University of Barcelona. All efforts were made to minimize animal suffering and to use no more than the number of animals needed for reliable scientific data.

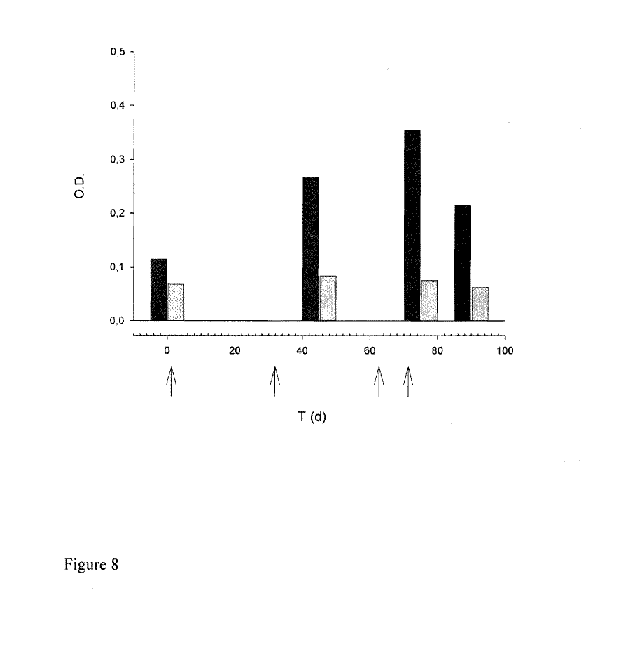

Dogs were subcutaneously immunized with either: a) 100 μg per animal of SEQ ID NO 3 conjugated with KLH and 1 mg Alum used as adjuvant (n=2) or b) 100 μg per animal of KLH and 1 mg Alum (no peptide n=2). The administered dose corresponds to a therapeutic amount from 0.00001 pg to 250 g of the antibody per animal. The immunization protocol consisted of a dose subcutaneously delivered up to four sites near the back of the neck, and repeated at days 31, 61, 75 (arrows in FIG. 8). Monitoring and supervising of the animals were performed weekly until day 100. Samples of serum were collected from each dog at days 0, 30, 60, 74 and 90. Each time, 5 ml blood samples were collected from the radial vein in plastic tubes with no additives. To isolate serum samples were kept at RT for 30 min, then were centrifuged for 10 min at 3000 rpm. Serum was collected, aliquoted and stored at −80° C. The titration of antibodies against SEQ ID NO 3 was performed by ELISA on 96-well plates developed by the biotin-avidin-peroxidase method as explained in Example 2. Plate sensitization was made overnight at 4° C. with 2 μg/ml of canine Abeta 1-40 peptide diluted in 50 mM NaHCO₃ pH 9.6.

No antibody was detected in any serum of control dogs (immunized with KLH and Alum) at any time-point (clear-bars of FIG. 8). In these animals, the highest values of optic density were found at day 60 but they were not different from background. At day 30 after the first immunization, a specific production of anti-Abeta antibodies was detected in serum samples of dogs injected with the Abeta 28-40 peptide (dark-bars in FIG. 8). The highest values of optic density were detected in samples of day 74.

These results clearly demonstrate that the Abeta 28-40 peptide injection in dogs induces a high specific immunogenic response to the canine Abeta peptide. Thus, the Abeta 28-40 peptide formulated in a standard veterinary vaccine composition suitable for administration to a canine subject provides an effective method to develop immunogenicity against Abeta peptide. These immunogens elicit the production of site-specific antibodies which bind to the soluble Abeta peptide, which immunoneutralizes the soluble Abeta-derived toxins and will facilitate the clearance of amyloid plaques in the dog CNS. No secondary effects were observed in the dogs, neither anaphylactic shocks, nor allergenic reactions, low of weight, etc.

Thus, the peptide sequences of this invention can be included in a veterinary vaccine formulation suitable for administration to a canine subject, with the aim of promoting an immune response with antibodies that can further immunoneutralize soluble Abeta proteins in the CNS for the prevention and treatment of CDS in the dog and other non-human mammals that have Abeta proteins with high homology to that of dogs, such as cats, monkeys, bears, rabbits, cows, sheep, pigs, guinea pigs, dolphins and Iberian lynxes.

### Example 10

### Evaluation of Cognitive Improvement and Abeta Plasma Levels of Aged Canine with CDS

Two female dogs (D1, golden retriever of 14 years old, 30 kg and D2, mongrel of 12 years, 9 kg), certified by their medical records from the veterinary hospital Ars Veterinaria of Barcelona, Spain, were used in this study. In both cases, inclusion of dogs and sample collection was formally approved by the owner. All animals were treated according to European legislation on animal handling and experiments (86/609/EU), and procedures were approved by the Ethical Committee of the University of Barcelona.

To evaluate the cognitive status of the dogs, a validated cognitive test was used in collaboration with pet owners. This test includes nine items: elimination behaviour, life rhythm, walking, posture, playful and exploratory behaviours, interaction with other animals or with the owners, learning of specific behaviours and adaptive capabilities and was filled out by a veterinarian. For each item score 1 indicated the normality of the specific behaviour, and scores 2, 3, 4 and 5 the degree of abnormal behaviour. The final total score reflected the cognitive status of the animal. In some cases normality was qualified considering the diagnostic previously established by the veterinarian The final total score reflects the cognitive status of the animal: a 16-79 score range allows the classification of the animals into three groups: 1) young control animals (YC); 2) dogs with light cognitive deficits (LCD); 3) dogs with severe cognitive deficits (SCD). Both dogs were evaluated before the beginning of the immunization protocol (day 0) and on day 71.

Both dogs were subcutaneously immunized with 100 μg of the peptide solution formed by 1/3 part SEQ ID NO 3 conjugated with KLH added to 2/3 of SEQ ID NO 1, and with 1 mg Alum used as adjuvant The immunization protocol consisted of a dose subcutaneously delivered near the back of the neck, and repeated at days 11, 21, 31 and 41.

Samples of serum were collected from each animal at days 0, 21, 31, 41 and 51. Each time, 5 ml blood samples were collected in plastic tubes with no additive. Blood samples were then kept at room temperature (RT) for 30 min., afterwards they were centrifuged for 10 min at 3000 rpm. Supernatants were collected as serum samples, aliquoted and stored at −80° C. Afterwards, Abeta 1-40/42 was detected in dog serum using the Amyloid 1-40/42 ELISA Kit detection (Immuno-Biological Laboratories, Co., Ltd, USA).

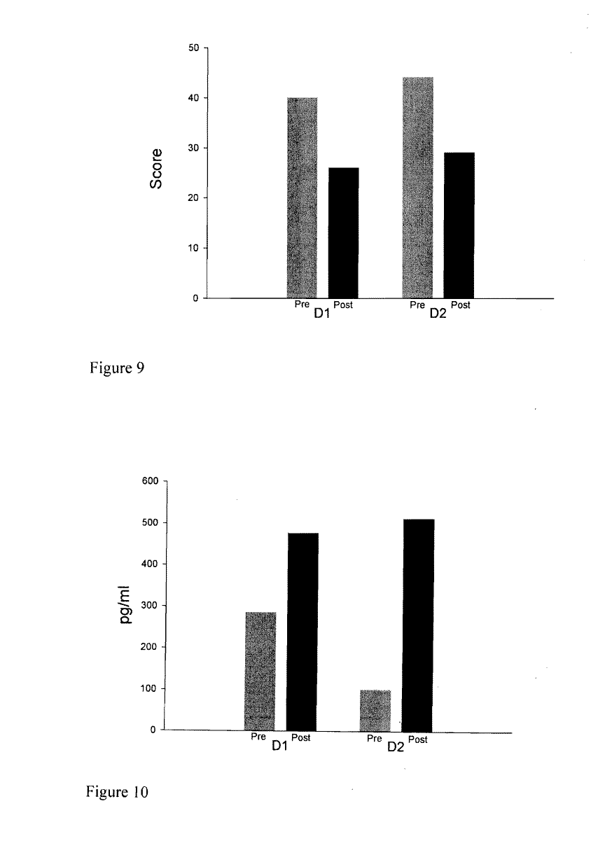

As shown in FIG. 9 each dog presented a 50% global cognitive improvement after the immunization. Thus, D1 preimmune score was 40 on day 0, and 26 on day 71 after the immunization process. D2 preimmune score on day 0 was 44 and 29 on day 71 after the immunization process.

The immunization process resulted in a modification of the levels of serum Abeta. As seen in FIG. 10), Abeta 1-40/42 soluble peptide increased in the two dogs on day 71 after immunization.

Taken together, the results herein presented clearly demonstrate that a standard pharmacological preparation of 1/3 SEQ ID NO 3 conjugated with KLH added to 2/3 of SEQ ID NO 1, formulated as a vaccine, induces a cognitive improvement in CDS dogs. In the same animals, this treatment also increased Abeta soluble serum levels, providing an effective method for the clearance of amyloid deposition, and improvement of amyloidosis related tissue degeneration.

### Example 11

### Application of Soluble Abeta Detection for Canine Cognitive Dysfunction Syndrome Diagnosis

As performed in Example 4 with the purified anti-Abeta 28-40 and anti-Abeta 1-40 antibodies and the canine Abeta peptide immobilized in a PVP membrane, other assays with serum of dogs suspected of suffering CDS are made, using the anti-Abeta 28-40 (or anti-SEQ ID NO 3) and anti-Abeta 1-40 (or anti-SEQ ID NO 1) antibodies as diagnostic agents. All the dogs in which a positive dot blot is detected are good diagnosed and suffer CDS.

The antibodies of the invention are included in a kit that contains all necessary ingredients, reagents and/or elements to perform the diagnostic method by detecting increased soluble Abeta in cerebrospinal fluid (CSF), blood and other peripheral fluids or tissue samples.

As has been demonstrated by way of these examples, the peptide sequence SEQ ID NO 1 and fragments thereof, i.e. SEQ ID NO 3, induce high immune responses without prejudicial secondary effects when administered in dogs suffering from CDS. Thus, the present invention results in an improvement of the current therapies used in the veterinarian field for curing those diseases, in which a cognition dysfunction exists due to the deposition of diffuse Abeta-plaques in the CNS of the animals.

1-19. (canceled)

20. A method for the treatment or prevention in a non-human mammal of a disease associated with amyloid deposition comprising the administration to said non-human animal of a agent which comprises a peptide consisting of the sequence of SEQ ID NO: 3 covalently coupled to an immunogenic carrier.

21. A method as defined in claim 20 wherein the carrier is keyhole limpet hemocyanine.

22. A method as defined in claim 20 wherein the agent further comprises a peptide having the sequence of SEQ ID NO:1,

23. A method as defined in claim 20 wherein the disease associated with amyloid deposition in the central nervous system is selected from the group of involutive depression, confusional syndrome, dysthymia, cognitive dysfunction syndrome (CDS) and amyloid-related tissue degeneration.

24. A method as defined in claim 20 wherein the non-human mammal is selected from the group consisting of dogs, cats, monkeys, bears, rabbits, cows, sheep, pigs, guinea pigs, dolphins and Iberian lynxes.

25. A conjugate comprising a peptide consisting of the sequence of SEQ ID NO:3 and an immunologically active carrier wherein the peptide and the carrier are covalently coupled.

26. A method for the treatment of an amyloid-related disease in a subject in need thereof which comprises the administration to said subject of a conjugate as defined in claim 25.

27. A method as defined in claim 25 wherein the amyloid-related disease is selected from the group of localised amyloidosis, systemic amyloidosis, secondary amyloidosis, scrapie, bovine spongiform encephalitis, Creutzfeld-Jacob disease, Alzheimer's disease, cerebral amyloid angiopathy, involutive depression, confusional syndrome, dysthymia and cognitive dysfunction syndrome (CDS) and amyloid-related tissue degeneration.

28. A composition comprising a first component and a second component selected from the group of
(i) a composition wherein the first component comprises a peptide consisting of the sequence of SEQ ID NO:3 and the second component comprises one or more peptides selected from the group of a peptide having a sequence of SEQ ID NO:10 and a functionally equivalent thereof,
(ii) a composition wherein the first component comprises a peptide consisting of the sequence of SEQ ID NO:3 and the second component comprises one or more peptides selected from the group of a peptide having a sequence of SEQ ID NO:10 and a functionally equivalent variant thereof and wherein the peptide forming part of the first component and/or the peptide or peptides forming part of the second component are covalently coupled to an immunologically active carrier and
(iii) a composition comprising a first component comprising a peptide consisting of the sequence of SEQ ID NO:3 and a second component comprising one or more peptides selected from the group of a peptide having the sequence of SEQ ID NO:10 and/or one or more functionally equivalent variants thereof wherein both the peptide forming the first component and the peptide or peptides forming the second component are covalently coupled to a single immunologically active carrier.

29. A composition as defined in claim 28 wherein the first component comprises a peptide consisting of the sequence SEQ ID NO: 3 and the second component comprises a peptide having the sequence of SEQ ID NO:1.

30. A composition as defined in claim 29 wherein a peptide consisting of the sequence of SEQ ID NO:3 is provided as a conjugate to an immunologically active carrier.

31. A method for the treatment of an amyloid-related disease in a subject in need thereof which comprises the administration to said subject of a composition according to claim 28.

32. A method as defined in claim 31 wherein the amyloid-related disease is selected from the group of localised amyloidosis, systemic amyloidosis, secondary amyloidosis, scrapie, bovine spongiform encephalitis, Creutzfeld-Jacob disease, Alzheimer's disease, cerebral amyloid angiopathy, involutive depression, confusional syndrome, dysthymia and cognitive dysfunction syndrome (CDS) and amyloid-related tissue degeneration.

33. A method of obtaining an antibody that specifically interacts with an amyloid peptide which comprises administering to a non-human mammal a composition comprising a peptide consisting of the sequence as defined in SEQ ID NO:3.

34. An antibody obtained by a method as defined in claim 33 or an antigen-binding fragment thereof.

35. A method for the diagnosis of a disease associated with amyloid deposition in a subject comprising contacting a biological sample from said subject with an antibody or antibody mixture wherein said antibody or antibody-mixture comprises an antibody raised against a peptide consisting of the sequence of SEQ ID NO:3 and detecting the formation of immune complexes between said antibody and at least an amyloid-containing component within the sample.

36. A method as defined in claim 35 wherein the biological sample is selected from the group of a biofluid or a sample from the central nervous system.

37. A method as defined in claim 35 wherein the sample from the central nervous system is a brain biopsy.

38. A method as defined in claim 35 wherein the biofluid is selected from the group of blood, serum and CSF.

39. A method as defined in claim 35 wherein the disease associated with amyloid depositions in the central nervous system is selected from the group of localised amyloidosis, systemic amyloidosis, secondary amyloidosis, scrapie, bovine spongiform encephalitis, Creutzfeld-Jacob disease, Alzheimer's disease, cerebral amyloid angiopathy, involutive depression, confusional syndrome, dysthymia, cognitive dysfunction syndrome (CDS) and amyloid-related tissue degeneration.


SEQUENCE LISTING (10 sequences)

SEQ ID NO: 1 - protein, 40 aa, H.sapiens
DAEFRHDSGYEVHHQKLVFFAEDVGSNKGAIIGLMVGGVV

SEQ ID NO: 2 - protein, 8 aa, H.sapiens
GLMVGGVV

SEQ ID NO: 3 - protein, 13 aa, H.sapiens
KGAIIGLMVGGVV

SEQ ID NO: 4 - protein, 9 aa, H.sapiens
IGLMVGGVV

SEQ ID NO: 5 - protein, 10 aa, H.sapiens
IIGLMVGGVV

SEQ ID NO: 6 - protein, 11 aa, H. sapiens
AIIGLMVGGVV

SEQ ID NO: 7 - protein, 12 aa, H.sapiens
GAIIGLMVGGVV

SEQ ID NO: 8 - protein, 14 aa, H.sapiens
NKGAIIGLMVGGVV

SEQ ID NO: 9 - protein, 15 aa, H.sapiens
SNKGAIIGLMVGGVV

SEQ ID NO: 10 - protein, 42 aa, H.sapiens [sequence omitted]